(19) United States
(12) Patent Application Publication
(10) Pub. No.: US 2012/0165207 A1
(43) Pub. Date: Jun. 28, 2012

(54) Methods for Monitoring Allograft Rejection

(75) Inventors: Atul J. Butte, Stanford, CA (US); Rong Chen, Fremont, CA (US); Minnie M. Sarwal, Portola Valley, CA (US); Tara Sigdel, Palo Alto, CA (US)

(73) Assignee: The Board of Trustees of the Leland Stanford Junior University, Palo Alto, CA (US)

(21) Appl. No.: 13/148,458

(22) PCT Filed: Feb. 12, 2010
(86) PCT No.: PCT/US2010/024023

§ 371 Date: Nov. 16, 2011

Related U.S. Application Data
(60) Provisional application No. 61/152,199, filed on Feb. 12, 2009.

(51) Int. Cl. G01N 33/566; C40B 30/04

(52) U.S. Cl. 506/9; 435/7.92; 435/7.1; 435/7.21

(57) ABSTRACT

Methods are provided for monitoring an allograft recipient for a rejection response, e.g., to predict, to diagnose, and/or to characterize a rejection response. In practicing the subject methods, the level of at least one protein in a sample from the allograft recipient, e.g., serum, urine, blood, CSF, tears or saliva, is evaluated, to monitor the subject. Also provided are compositions, systems, and kits that find use in practicing the subject methods.

### CROSS REFERENCE TO RELATED APPLICATIONS

Pursuant to 35 U.S.C. §119 (e), this application claims priority to the filing date of the U.S. Provisional Patent Application Ser. No. 61/152,199 filed Feb. 12, 2009; the disclosure of which are herein incorporated by reference.

### GOVERNMENT RIGHTS

This invention was made with Government support under contracts R01 GM079719 and K22 LM008261 and R01 AI61739 and U01 AI55795 awarded by the National Institutes of Health. The Government has certain rights in this invention.

### BACKGROUND

Protein biomarkers are used in the clinic to predict the onset of disease, diagnose it, monitor its progression, and provide prognosis as to its responsiveness to therapeutics. Examples of biomarkers include prostate specific antigen (PSA) for prostate cancer and carcinoembryonic antigen for gastrointestinal cancer. Protein biomarkers are theoretically better than mRNA markers because of their increased stability and the broader range of technologies available to study them. However, they are also harder to find. The use of serum proteomics as a way to find diagnostic biomarkers has received considerable attention and investment with limited success in the last decade due to the limited sensitivity and dynamic range of mass spectrometers to analyze a clinical biopsy-sized sample.

The field of solid organ transplantation is in urgent need of diagnostic protein biomarkers. Acute and chronic rejections are frequent after all types of solid organ transplants. This situation is treated by short term increases in immunosuppressive therapy but can eventually lead to graft loss.

Furthermore, non-invasive methods for predicting or diagnosing rejection and guiding optimal titration of immunosuppressive therapy are critically needed for transplant patients. Currently, diagnosis of acute rejection (AR) requires a tissue biopsy, which is an invasive procedure whose usefulness is limited by the risk of complications and cost, tissue sampling, as well as the molecular heterogeneity in the transcriptional signals in tissue rejection. These factors discourage biopsy use for frequent serial monitoring of rejection.

Some efforts have been made to identify organ-specific AR protein markers. For example, in renal transplant rejection, VEGF has been studied in serum and urine, and CXCL9 and CXCL10 have been examined in urine. However, very few transplant biomarkers have been validated extensively and applied in clinical settings. Moreover, none has been shown to work universally across different transplanted organs.

### RELEVANT LITERATURE

Publications of interest include Peng, W., et al. (2008) Acute renal allograft rejection is associated with increased levels of vascular endothelial growth factor in the urine. Nephrology 13:73-79; Peng, W., et al. (2008) Prediction of subclinical renal allograft rejection by vascular endothelial growth factor in serum and urine. Journal of nephrology 21: 535-542 (2008); and Hauser, I. A., et al. Prediction of acute renal allograft rejection by urinary monokine induced by IFN gamma (MIG). J Am Soc Nephrol 16, 1849-1858 (2005).

### SUMMARY OF THE INVENTION

Methods are provided for monitoring an allograft recipient for a rejection response. In practicing the subject methods, a sample, e.g. a blood sample, from a subject who has received an allograft is evaluated for the protein level of at least one phenotype determinative gene to obtain a protein level determination. The obtained protein level determination is then employed to monitor the subject. Also provided are systems and kits that find use in practicing the subject methods. The subject methods find use in a variety of applications, including predicting the onset of a rejection response, diagnosing a rejection response, and characterizing a rejection response.

In some aspects of the present invention, a method of monitoring an allograft recipient for a rejection response is provided, in which the protein level of at least one phenotype determinative gene in a sample from the allograft recipient is measured to obtain a protein level determination, and the protein level determination is employed to monitor the individual for a rejection response. In some embodiments, the sample is serum, urine, blood, CSF, tears or saliva. In particular embodiments, the sample is serum. In some embodiments, the phenotype determinative gene is one or more of the following: CXCL9, PECAM1, and/or CD44. In certain embodiments, the phenotype determinative gene is PECAM1 and/or CD44. In some embodiments, the rejection response is an acute rejection. In some embodiments, the protein level determination is compared to a reference profile. In some such embodiments, the reference profile is a protein level determination of a phenotype determinative gene from an individual that has not received an allograft, a protein level determination of a phenotype determinative gene from a stable allograft recipient, or a protein level determination of a phenotype determinative gene from an allograft recipient undergoing a rejection response.

In some aspects of the present invention, systems for monitoring an allograft recipient for a rejection response are provided, where the system includes a protein level determination element for measuring the level of at least one protein of a phenotype determinative gene in a sample from the allograft recipient to obtain a protein level determination; and a phenotype determination element for employing the protein level determination to monitor the allograft recipient for a rejection response. In some embodiments, the protein level determination element comprises at least one reagent for assaying a sample for the at least one protein. In some embodiments, the phenotype determination element is a sample from an allograft recipient undergoing a rejection response, a sample from a stable allograft recipient, or a sample from an individual that did not receive an allograft. In some embodiments, the phenotype determination element is a reference profile prepared from an allograft recipient undergoing a rejection response, a stable allograft recipient, or an individual that did not receive an allograft. In certain embodiments, the sample is serum, urine, or blood. In certain embodiments, the protein is one or more of the following: CXCL9, PECAM1 and/or CD44. In certain embodiments, the rejection response is an acute rejection.

In some aspects of the present invention, kits for monitoring an allograft recipient for a rejection response are provided, where the kit includes a protein level determination element for measuring the level of at least one protein in a sample from the allograft recipient to obtain a protein level determination; and a phenotype determination element for employing the protein level determination to monitor the allograft recipient for a rejection response. In some embodiments, the protein level determination element comprises at least one reagent for assaying a sample for the at least one protein (e.g., one or more antibody specific for the protein(s) being assayed). In some embodiments, the phenotype determination element is a sample from an allograft recipient undergoing a rejection response, a sample from a stable allograft recipient, or a sample from an individual that did not receive an allograft. In some embodiments, the phenotype determination element is a reference profile prepared from an allograft recipient undergoing a rejection response, a stable allograft recipient, or an individual that did not receive an allograft. In some embodiments, the sample is serum, urine, or blood. In certain embodiments, the protein is one or more of the following: CXCL9, PECAM1 and/or CD44. In some embodiments, the rejection response is an acute rejection.

Also provided are reference profiles of protein levels for a phenotype that is one of an individual that has not received an allograft; a stable allograft recipient; and/or an allograft recipient undergoing a rejection response.

### BRIEF DESCRIPTION OF THE FIGURES

FIG. 1 depicts how protein biomarkers were more likely found with three or more gene expression data sets. The association p_value between predicted and known protein biomarkers for 41 diseases (Table 1) was calculated. Then, the −log 10 (p-value) was compared between the 11 diseases for which we have ≧3 gene expression data sets and 30 diseases for which we have <3 data sets using notched box plots in R. The 11 diseases with ≧3 data yielded a p_value of 5×10⁻⁴ at the median, 2×10⁻⁵ at the 75% ile, and 0.02 at 25% ile. The 30 diseases with <3 data yielded a p_value of 0.12 at the median, 0.004 at the 75% ile, and 1 at 25% ile. Known protein biomarkers were statistically significantly more enriched in differentially expressed genes in diseases with ≧3 datasets than disease with <3 datasets (p=0.004, Mann-Whitney U), indicating that protein biomarkers are more likely identified with integration methods employing 3 or more datasets.

FIG. 2 depicts a schematic for the identification of cross-organ AR protein biomarkers through integration of gene expression data. Three microarray studies that examined gene expression after rejection in biopsy samples from pediatric renal, adult renal, and adult heart transplants (the latter two from NCBI Gene Expression Omnibus) were integrated. 45 genes were identified that were upregulated in common in acute rejection (AR) as compared to stable graft function. Among ten proteins tested by ELISA, three showed statistically significantly higher protein concentration in serum samples with AR, compared to stable samples after renal transplant. The same three proteins showed significantly higher concentration in AR samples from cardiac transplantation. Immunohistochemistry showed that our novel protein PECAM1 was increased in AR vs. stable biopsies in renal, hepatic and cardiac transplantation. All three biomarkers were from our identified AR pathway, and two of them showed detectable protein abundance in the Biofluid proteome database we constructed before.

FIG. 3: Serum ELISA results of three protein biomarkers in renal transplantation. We measured the protein concentration of ten genes by ELISA in independent serum samples of 19 acute rejection (AR) patients and 20 patients with stable (STA) graft function after renal transplant. PECAM1 (a), CXCL9 (b), and CD44 (c) have statistically significantly higher protein concentration in the AR serum samples, compared to stable samples. The notched boxplots were plotted using boxplot function in R package. P-values were calculated using Mann-Whitney U test. (d) In ROC curves used to distinguish AR from STA, the areas under the curves were 0.811, 0.864, and 0.761 for PECAM1, CXCL9, and CD44, respectively.

FIG. 4: Plasma ELISA results of three protein biomarkers in cardiac transplantation. We measured the protein concentration of PECAM1 (a), CXCL9 (b) and CD44 (c) by ELISA in the plasma of 32 acute rejections (AR) and 31 stable (STA) patients after cardiac transplantation. All three proteins have statistically significantly higher concentration in the AR serum samples, compared to STA in the notched box plots. P-values were calculated using Mann-Whitney U test. (d) In ROC curves used to distinguish AR from STA, the areas under the curves were 0.716, 0.672, and 0.711 for PECAM1, CXCL, and CD44, respectively.

FIG. 5: Immunohistochemistry of PECAM1 between AR and Stable in renal, hepatic, and cardiac allograft biopsies. (a) Acute rejection in renal allograft biopsy with PECAM1 positive infiltrating lymphocytes and monocytes; endothelial cell staining is seen in glomeruli and peritubular capillaries as well. (b) Renal allograft protocol biopsy (stable graft function) with only endothelial cell staining in glomeruli and peritubular capillaries. Similarly, dense staining was observed in the AR tissues after hepatic (c) and cardiac (e) transplants, in infiltrating mononuclear cells and endothelial cells of capillaries and larger blood vessels. In hepatic (d) and cardiac (f) transplant biopsies from stable grafts, only weak endothelial cell staining was identified (magnification ×400).

FIG. 6: Histogram of overlapping genes in three transplant rejection microarray data after shuffling gene labels. We shuffled the gene labels in the three pediatric renal, adult renal and cardiac transplant rejection gene expression data sets, calculated differentially expressed AR genes in common. After repeating the processed 100,000 times, we plotted the distribution of the number of overlapping genes (histogram bars). The probability of getting ≧17 common genes by random is less than 1% and the probability of getting ≧24 common genes is less than 1×10-5 (curve overlay).

FIG. 7: A cross-organ transplant rejection pathway. Among 45 genes that were upregulated in the AR compared with stable biopsy samples across transplanted organs, 23 of them were involved in a single pro-inflammatory pathway regulated by STAT-1. We tested 5 proteins (circled) from the pathway by ELISA, and three of them (solid circle) were validated as cross-organ serum protein biomarkers for transplant rejection. The untested 18 AR proteins from the 45 are highlighted in the pathway, providing promising leads for further validation. Five of them (solid star) were known to have detectable levels of protein expression in the normal serum or urine according to our human biofluid proteome database (Dudley, J. T. & Butte, A. J. (2009) Pac Symp Biocomput, 27-38). Seven of them (clear star) were studied in knock-out mouse models (Eppig, J. T., Blake, J. A., Bult, C. J., Kadin, J. A. & Richardson, J. E. (2007) Nucleic Acids Res 35, D630-637), confirming their involvement in the immune system.

FIG. 8: ROC curves of predicting renal and cardiac transplant rejection using PECAM1+CXCL9. ROC curves showed three-fold cross-validation results on predicting renal (solid curve) and cardiac (dotted curve) transplant rejection (AR) from stable graft function using a combined panel of PECAM1 and CXCL9 proteins in serum (renal) and plasma (cardiac). The true positive rates were showed as mean±standard error across 1000 three-fold cross-validation. It showed a slight improvement over individual proteins on cardiac AR and no improvement on renal AR.

### DESCRIPTION OF THE SPECIFIC EMBODIMENTS

Methods are provided for monitoring an allograft recipient for a rejection response. In practicing the subject methods, the expression of at least one phenotype determinant gene in a sample from the subject, e.g., a serum sample, is assayed to obtain an expression evaluation, e.g. protein level, for the at least one gene. The obtained expression evaluation is then employed to monitor whether the allograft is being rejected. Also provided are compositions, systems and kits that find use in practicing the subject methods. The methods and compositions find use in a variety of applications, including predicting the onset of a rejection response, diagnosing a rejection response, and characterizing a rejection response.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, the subject invention is directed to methods of monitoring an allograft recipient for a rejection response, as well as reagents and kits for use in practicing the subject methods. In further describing the invention, the subject methods are described first, followed by a review of the reagents, systems and kits for use in practicing the subject methods.

### Methods of Monitoring an Allograft Recipient for a Rejection Response

The subject invention provides methods of monitoring an allograft recipient for a rejection response. An allograft is a graft organ, tissue or cell(s) that has been introduced into/onto a subject. Accordingly, an allograft recipient is a subject that has received a graft organ, tissue or cell(s). An allograft recipient may have received the graft organ, tissue or cell(s) about one hour prior to monitoring, about one day prior to monitoring, about one week prior to monitoring, about two weeks prior to monitoring, about one months prior to monitoring, about two months prior to monitoring, about four months prior to monitoring, about eight months prior to monitoring, about one year prior to monitoring, about two years prior to monitoring, about five years prior to monitoring, about ten years prior to monitoring, about fifteen years prior to monitoring, etc. A variety of tissue and organ types can be transplanted, including but not limited to skin, cornea, heart, liver, kidney, bone, bone marrow, ligament, and tendon.

In a rejection response, the allograft recipient's immune system rejects the allograft that has been introduced into/onto the recipient. In other words, the allograft recipient does not tolerate or maintain the organ, tissue or cell(s) that has been transplanted to it. Rejection by the immune system of a tissue transplant generally occurs when the transplanted tissue is immunologically foreign.

A rejection response can be an acute rejection response. “Acute rejection” or “AR” is characterized by infiltration of the transplanted tissue by immune cells of the recipient, which carry out their effector function and destroy the transplanted tissue. The onset of acute rejection is rapid and generally occurs in humans within a few weeks after transplant surgery. Generally, acute rejection can be inhibited or suppressed with immunosuppressive drugs such as rapamycin, cyclosporin A, anti-CD40L monoclonal antibody and the like.

A rejection response can also be a chronic rejection response. “Chronic rejection” or “CR” generally occurs in humans within several months to years after engraftment, even in the presence of successful immunosuppression of acute rejection. Fibrosis is a common factor in chronic rejection of all types of organ transplants. Chronic rejection can typically be described by a range of specific disorders that are characteristic of the particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis; in kidney transplants, such disorders include, obstructive nephropathy, nephrosclerorsis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs.

The term “allograft rejection” or “transplant rejection” encompasses both acute and chronic transplant rejection. An “allograft rejection response” or “transplant rejection response” or “rejection response” comprises all aspects of the rejection response by the body to the allograft, i.e. all aspects of the body's response to an allograft in which the body rejects the allograft. By “monitoring an allograft recipient for a rejection response” is meant predicting the onset of a rejection response, diagnosing the presence or absence of a rejection response, and/or characterizing a rejection response.

In practicing the subject methods, a subject or patient sample, e.g., cells or fluid thereof, e.g., blood or serum, is assayed to determine whether the allograft recipient from which the assayed sample was obtained is subject to an allograft rejection response, i.e., will have or is having a rejection response to an allograft. Accordingly, the first step of the subject methods is to obtain a suitable sample from the subject or patient of interest, i.e., a patient having at least one allograft. The sample is derived from any initial suitable source, where sample sources of interest include, but are not limited to, many different physiological sources, e.g., blood, serum, urine, saliva, tears, CSF, or tissue derived samples. In certain embodiments, a suitable initial source for the patient sample is serum. As such, the sample employed in the subject assays of these embodiments is generally a serum-derived sample. In other embodiments, a suitable initial source is urine. As such, the sample employed in the subject assays of these embodiments is generally a urine-derived sample. Any convenient protocol for obtaining such samples may be employed, where suitable protocols are well known in the art.

In practicing the subject methods, the sample is evaluated, to obtain an expression evaluation, e.g. an expression profile, for one or more genes, i.e. phenotype determinative genes, where the term expression profile is used broadly to include a gene expression profile, that is, the determination of the expression of one or more genes at the RNA or protein level. By “phenotype determinative genes”, it is meant genes that are differentially expressed or present at different levels in allograft recipients undergoing a rejection response versus stable allograft recipients or individuals that have not received an allograft.

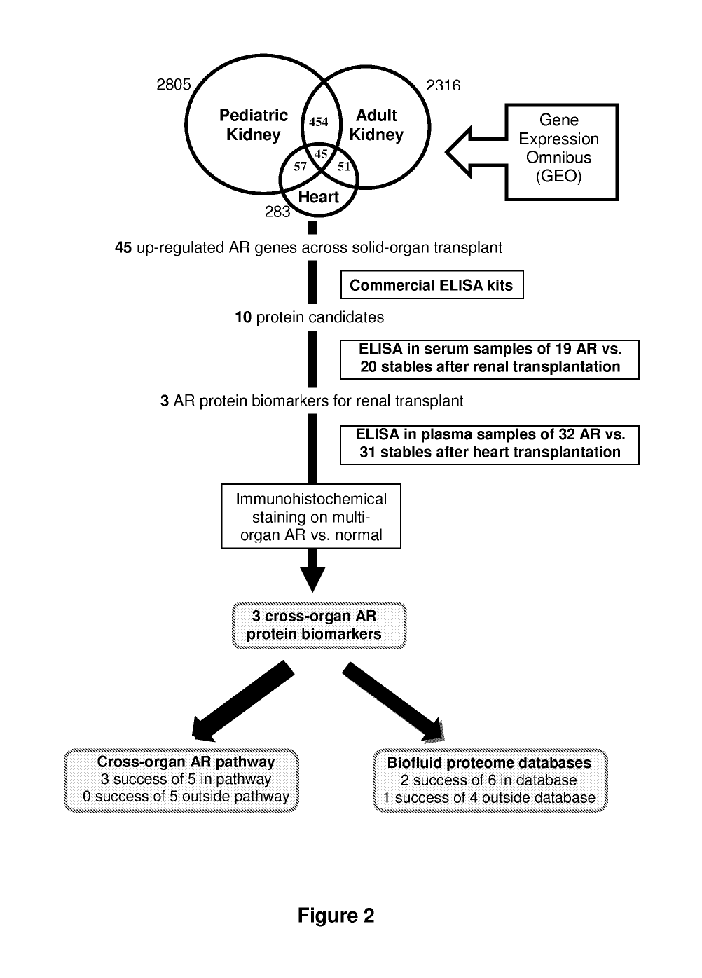

In some embodiments, the phenotype determinative genes of interest may be identified by comparing the expression profiles of one or more genes from two or more studies and identifying those genes that are commonly upregulated or down-regulated amongst all studies. See, for example, FIG. 2. The changes in gene regulation that are observed amongst all datasets may be validated by a second quantitative method, for example, qRT-PCR, ELISA, xMAP technology. The expression levels of the validated genes may then be assayed in tissue, e.g. transplanted tissue, or body fluid, e.g. serum, urine, blood, CSF, saliva, tears, etc., to identify genes of interest for use in the present invention.

In some embodiments, the phenotype determinative genes include, but are not limited to, the 45 genes provided in Table 2 of the Examples section. Note that for the genes listed in Table 2, detailed information for each specific gene, including nucleotide sequence information, can be retrieved through the NCBI Entrez nucleotide database at located at the website: http(colon)//www(dot)ncbi.nlm.nih(dot)gov/ by selecting “Gene” as the database and entering the Entrez Gene ID number listed into the search window. In certain embodiments, the expression level of genes other than those listed in Table 2 is also evaluated.

In some embodiments, the phenotype determinative genes of interest include PECAM1, CXCL9, and/or CD44. “PECAM1” (platelet/endothelial cell adhesion molecule 1), which is also known as CD31, refers to a gene of the nucleic acid sequence described in Genbank Accession No. NM_{—}000442 (SEQ ID NO:1). The protein encoded by the PECAM1 gene is an integral membrane protein (SEQ ID NO:2). “CXCL9” (chemokine (C-X-C motif) ligand 9), which is also known as MIG, CMK, crg-10, and Scyb9, refers to a gene of the nucleic acid sequence described in Genbank Accession No. NM_{—}002416 (SEQ ID NO:3). The protein encoded by the CXCL9 gene is a chemokine, i.e. a secreted protein (SEQ ID NO:4). “CD44”, which is also known as HERMES, Ly-24, MDU and Pgp-1, refers to a gene of the nucleic acid sequence described in Genbank Accession No. NM_{—}000610 (isoform 1, SEQ ID NO:5), NM_{—}001001389 (isoform 2, SEQ ID NO:7), NM_{—}001001390 (isoform 3, SEQ ID NO:9), NM_{—}001001391 (isoform 4, SEQ ID NO:11), or NM_{—}001001392 (SEQ ID NO:13). The proteins encoded by the CD44 gene are integral membrane proteins (SEQ ID NO:6, SEQ ID NO:8, SEQ ID NO:10, SEQ ID NO:12, SEQ ID NO:14).

The terms “evaluating”, “assaying”, “measuring”, “assessing,” and “determining” are used interchangeably to refer to any form of measurement, including determining if an element is present or not, and including both quantitative and qualitative determinations. Assessing may be relative or absolute. “Assessing the presence of” includes determining the amount of something present, as well as determining whether it is present or absent.

As used herein, the term “gene” or “recombinant gene” refers to a nucleic acid comprising an open reading frame encoding a polypeptide, including exon and (optionally) intron sequences. The term “intron” refers to a DNA sequence present in a given gene that is not translated into protein and is generally found between exons in a DNA molecule. In addition, a gene may optionally include its natural promoter (i.e., the promoter with which the exons and introns of the gene are operably linked in a non-recombinant cell, i.e., a naturally occurring cell), and associated regulatory sequences, and may or may not have sequences upstream of the AUG start site, and may or may not include untranslated leader sequences, signal sequences, downstream untranslated sequences, transcriptional start and stop sequences, polyadenylation signals, translational start and stop sequences, ribosome binding sites, and the like.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from viral, procaryotic or eukaryotic mRNA, genomic DNA sequences from viral, procaryotic or eukaryotic DNA, and even synthetic DNA sequences. A transcription termination sequence may be located 3′ to the coding sequence.

The term “nucleic acid” includes DNA, RNA (double-stranded or single stranded), analogs (e.g., PNA or LNA molecules) and derivatives thereof. The terms “ribonucleic acid” and “RNA” as used herein mean a polymer composed of ribonucleotides. The terms “deoxyribonucleic acid” and “DNA” as used herein mean a polymer composed of deoxyribonucleotides. The term “mRNA” means messenger RNA. An “oligonucleotide” generally refers to a nucleotide multimer of about 10 to 100 nucleotides in length, while a “polynucleotide” includes a nucleotide multimer having any number of nucleotides.

The terms “protein” and “polypeptide” as used in this application are interchangeable. “Polypeptide” refers to a polymer of amino acids (amino acid sequence) and does not refer to a specific length of the molecule. Thus peptides and oligopeptides are included within the definition of polypeptide. This term also refers to or includes post-translationally modified polypeptides, for example, glycosylated polypeptide, acetylated polypeptide, phosphorylated polypeptide and the like. Included within the definition are, for example, polypeptides containing one or more analogs of an amino acid, polypeptides with substituted linkages, as well as other modifications known in the art, both naturally occurring and non-naturally occurring.

A determination of the expression of one or more genes, i.e. the obtainment of an expression evaluation, may be made by measuring nucleic acid transcripts, e.g. mRNAs, of the one or more genes of interest, e.g. a genomic expression profile; or by measuring levels of one or more different proteins/polypeptides that are expression products of one or more genes of interest, i.e. a protein level determination e.g. a proteomic expression profile. In other words, the term “expression evaluation” is used broadly to include a gene expression profile, that is, the determination of the expression of one or more genes at the RNA level or protein level.

In some embodiments, expression of genes may be evaluated by obtaining a nucleic acid expression profile, i.e. an RNA level determination, where the amount or level of one or more nucleic acids in the sample is determined, e.g., the nucleic acid transcript of the one or more genes of interest. In these embodiments, the sample that is assayed to generate the expression profile is a nucleic acid sample. The nucleic acid sample includes a plurality or population of distinct nucleic acids that includes the expression information of the phenotype determinative genes of interest of the cell or tissue being diagnosed. The nucleic acid may include RNA or DNA nucleic acids, e.g., mRNA, cRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from a cell or tissue harvested from a subject to be diagnosed, e.g., via a blood draw or biopsy of tissue, using standard protocols, where cell types or tissues from which such nucleic acids may be generated include any tissue in which the expression pattern of the to be determined phenotype exists, including, but not limited to, peripheral blood lymphocyte cells, etc., as reviewed above.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array-based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Alternatively, non-array based methods for quantitating the level of one or more nucleic acids in a sample may be employed, including those based on amplification protocols, e.g., Polymerase Chain Reaction (PCR)-based assays, including quantitative PCR, reverse-transcription PCR (RT-PCR), real-time PCR, and the like.

In some embodiments, expression of genes may be evaluated by obtaining a protein level profile, i.e. a protein level determination, where the amount or level of one or more proteins/polypeptides in the sample is determined, e.g., the protein/polypeptide encoded by the gene of interest. In these embodiments, the sample that is assayed to generate the expression profile employed in the diagnostic methods is a protein sample. Where the expression profile is a protein level determination, i.e. a protein level profile, i.e. a profile of one or more protein levels in a sample, any convenient protocol for evaluating protein levels may be employed wherein the level of one or more proteins in the assayed sample is determined.

While a variety of different manners of assaying for protein levels are known in the art, one representative and convenient type of protocol for assaying protein levels is ELISA. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hrs at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hr at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatograpic column or filter with a wash solution or solvent.

Alternatively, non-ELISA based-methods for measuring the levels of one or more proteins in a sample may be employed. Representative examples include but are not limited to mass spectrometry, proteomic arrays, xMAP™ microsphere technology, flow cytometry, western blotting, and immunohistochemistry.

The resultant data provides information regarding expression for each of the genes that have been probed, wherein the expression information is in terms of whether or not the gene is expressed and, typically, at what level, and wherein the expression data may be both qualitative and quantitative.

In generating the expression profile, in some embodiments a sample is assayed to generate an expression profile that includes expression data for at least one gene/protein, sometimes a plurality of genes/proteins, where by plurality is meant at least two different genes/proteins, and often at least about 3, typically at least about 10 and more usually at least about 15 different genes/proteins or more, such as 50 or more, or 100 or more, etc.

In the broadest sense, the expression evaluation may be qualitative or quantitative. As such, where detection is qualitative, the methods provide a reading or evaluation, e.g., assessment, of whether or not the target analyte, e.g., nucleic acid or expression product, is present in the sample being assayed. In yet other embodiments, the methods provide a quantitative detection of whether the target analyte is present in the sample being assayed, i.e., an evaluation or assessment of the actual amount or relative abundance of the target analyte, e.g., nucleic acid or protein in the sample being assayed. In such embodiments, the quantitative detection may be absolute or, if the method is a method of detecting two or more different analytes, e.g., target nucleic acids or protein, in a sample, relative. As such, the term “quantifying” when used in the context of quantifying a target analyte, e.g., nucleic acid(s) or protein(s), in a sample can refer to absolute or to relative quantification. Absolute quantification may be accomplished by inclusion of known concentration(s) of one or more control analytes and referencing the detected level of the target analyte with the known control analytes (e.g., through generation of a standard curve). Alternatively, relative quantification can be accomplished by comparison of detected levels or amounts between two or more different target analytes to provide a relative quantification of each of the two or more different analytes, e.g., relative to each other.

In certain embodiments the expression of only one gene is evaluated. In yet other embodiments, the expression of two or more, e.g., about 3 or more, about 10 or more, about 15 or more, about 25 or more, or about 45 genes is evaluated. Accordingly, in the subject methods, the expression of at least one gene in a sample is evaluated. In certain embodiments, the evaluation that is made may be viewed as an evaluation of the transcriptosome, as that term is employed in the art.

Following obtainment of the expression profile, e.g. a protein level determination, from the sample being assayed, the expression profile is employed to monitor the allograft recipient for a rejection response, e.g. to predict graft rejection, diagnose graft rejection, or characterize graft rejection. In some embodiments, the protein level determination is employed directly to make a prediction, diagnosis, or characterization, e.g. without comparison to a phenotype determination element. For example, a protein level determination may be obtained by measuring the absolute concentration of protein in a defined volume of sample. For example, an allograft recipient may be diagnosed as undergoing a rejection response if the concentration of PECAM1 in the recipient's serum is about 4 ng/ml or greater, e.g. 4 ng/ml-12 ng/ml, e.g. 4 ng/ml, 4.3 ng/ml, 6 ng/ml, 7.5 ng/ml, 10 ng/ml, or 12 ng/ml; if the concentration of CXCL9 in recipient's serum is about 0.8 ng/ml or greater, e.g. 0.8 ng/ml-5.3 ng/ml, e.g. 0.8 ng/ml, 1 ng/ml, 1.5 ng/ml, 2 ng/ml, or 5.3 ng/ml; or the concentration of CD44 in the recipient's serum is about 300 ng/ml or greater, e.g. 300 ng/ml-500 ng/ml, e.g. 300 ng/ml, 320 ng/ml, 350 ng/ml, 400 ng/ml, or 450 ng/ml. Likewise, an allograft recipient may be diagnosed as undergoing a rejection response if the concentration of PECAM1 in the recipient's plasma is about 2.5 ng/ml or greater, e.g. 2.5 ng/ml-12 ng/ml, e.g. 2.5 ng/ml, 4 ng/ml, 5 ng/ml, 6.5 ng/ml, 9 ng/ml, or 12 ng/ml; if the concentration of CXCL9 in the recipient's plasma is about 0.05 ng/ml or greater, e.g. 0.05 ng/ml-0.7 ng/ml, e.g. 0.05 ng/ml, 0.1 ng/ml, 0.15 ng/ml, 0.2 ng/ml, 0.3 ng/ml, 0.4 ng/ml, 0.6 ng/ml, or 0.7 ng/ml; or if the concentration of CD44 in the recipient's plasma is about 225 ng/ml or greater, e.g. 225 ng/ml-550 ng/ml, e.g. 225 ng/ml, 250 ng/ml, 325 ng/ml, 450 ng/ml, or 550 ng/ml.

In some embodiments, the expression profile, e.g. protein level determination, is employed by comparing the expression profile with a phenotype determination element, i.e. a “reference element,” or “control element”, to identify similarities or differences with the phenotype determination element, where the similarities or differences that are identified are then employed to monitor the allograft recipient from which the sample was obtained/derived. The terms “phenotype determination element”, “reference element” and “control element” are used herein to refer to an element that is characteristic of a particular phenotype that may be observed in a patient, for example, an allograft patient undergoing a rejection response, a stable allograft patient, or an individual that has not received an allograft. For example, a phenotype determination element may be a reference, or control, profile. A reference/control profile is a standardized pattern of gene expression or levels of expression of certain genes to be used to interpret the expression profile of a given allograft recipient. The reference/control profile may be a profile that is obtained from a sample from an allograft recipient that is undergoing a rejection response; such a reference profile would be a positive reference/control profile. Other positive reference/control profiles would include but are not limited to profiles obtained from an allograft recipient undergoing an acute rejection (AR) response or a chronic rejection (CR) response. Alternatively, the reference/control profile may be from a sample from a stable allograft recipient, or an individual that did not receive an allograft; such reference profiles would be negative reference/control profiles. Reference/control profiles are preferably obtained from the same type of sample as the sample that was employed to generate the expression profile for the allograft recipient being monitored. For example, if the serum of an allograft recipient is being evaluated, the reference/control profile would preferably be of serum.

In certain embodiments, the obtained expression profile is compared to a single reference/control profile to obtain information regarding the status of the allograft recipient being monitored. In certain embodiments, the obtained expression profile is compared to two or more reference/control profiles. For example, the obtained expression profile may be compared to a negative reference profile and a positive reference profile to obtain confirmed information regarding if the allograft recipient is undergoing a rejection response. Alternatively, the obtained expression profile may be compared to a positive AR reference profile and a positive CR reference profile to obtain confirmed information regarding if the allograft recipient is undergoing an acute rejection response or a chronic rejection response. Thus, one can assay for a variety of graft-related pathologies, e.g., chronic rejection (or CAN) and/or drug toxicity (DT) (see, e.g., U.S. patent application Ser. No. 11/375,681, filed on Mar. 3, 2006, which is incorporated by reference herein in its entirety).

The comparison of the obtained expression profile and the one or more reference/control profiles may be performed using any convenient methodology, where a variety of methodologies are known to those of skill in the art. For example, those of skill in the art of arrays will know that array profiles may be compared by, e.g., comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above. Similarly, those of skill in the art of ELISAs will know that ELISA data may be compared by, e.g. normalizing to standard curves, comparing normalized values, etc.

The comparison step results in information regarding how similar or dissimilar the obtained expression profile is to the control/reference profile(s), which similarity/dissimilarity information is employed to monitor an allograft recipient, for example to predict the onset of a rejection response, diagnose a rejection response, or characterize a rejection response. For example, similarity with a positive reference/control indicates that the allograft recipient is undergoing an allograft rejection. Likewise, similarity with a negative control indicates that the allograft recipient is not undergoing an allograft rejection. Similarity may be based on relative expression levels, absolute expression levels or a combination of both. Similarity may be determined by comparison to both positive and negative control references. In certain embodiments, a similarity determination is made using a computer having a program stored thereon that is designed to receive input for a gene level expression result obtained from a subject, e.g., from a user, determine similarity to one or more reference profile, and return an allograft status result (or rejection response), e.g., to a user (e.g., lab technician, physician, allograft recipient, etc.). Further descriptions of computer-implemented aspects of the invention are described below.

Depending on the type and nature of the reference/control profile(s) to which the obtained expression profile is compared, the above comparison step yields a variety of different types of information regarding the cell/bodily fluid that is assayed. As such, the above comparison step can yield a positive/negative prediction of the onset of a rejection response. Alternatively, such a comparison step can yield a positive/negative diagnosis of a rejection response. Alternatively, such a comparison step can provide a characterization of a rejection response.

In some embodiments, other analysis may be employed in conjunction with the aforementioned expression level determination to monitor the allograft recipient for a rejection response. Such analyses are well known in the art, and include, for example, an analysis of a tissue biopsy for the presence of lymphocytes, e.g. T-cells, infiltrating the transplanted tissue, in some instances accompanied by a heterogeneous collection of other cell types including eosinophils, plasma cells and neutrophils, the proportions of which are useful in diagnosing the exact type of rejection; evidence of structural injury to the transplanted tissue; injury to the blood vessels in the transplanted tissue; symptoms of organ failure; and the like.

In some embodiments, monitoring an allograft recipient includes providing a prediction, diagnosis, or characterization of a rejection response. In such embodiments, the prediction, diagnosis, or characterization may be provided by providing, i.e. generating, a written report that includes the artisan's monitoring assessment, i.e. the artisan's prediction of the onset of a rejection response (a “rejection prediction”), the artisan's diagnosis of the subject's rejection response (a “rejection diagnosis”), or the artisan's characterization of the subject's rejection response (a “rejection characterization”). Thus, a subject method may further include a step of generating or outputting a report providing the results of a monitoring assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a subject monitoring assessment and its results. A subject report includes at least a rejection prediction, rejection diagnosis, or rejection characterization, i.e. a prediction as to the likelihood of a patient developing a rejection response, a diagnosis of a rejection response, or a characterization of a rejection response, respectively. A subject report can be completely or partially electronically generated. A subject report can further include one or more of: 1) information regarding the testing facility; 2) service provider information; 3) patient data; 4) sample data; 5) an assessment report, which can include various information including: a) reference values employed, and b) test data, where test data can include, e.g., a protein level determination; 6) other features.

The report may include information about the testing facility, which information is relevant to the hospital, clinic, or laboratory in which sample gathering and/or data generation was conducted. Sample gathering can include obtaining a fluid sample, e.g. blood, saliva, urine etc.; a tissue sample, e.g. a tissue biopsy, etc. from a subject. Data generation can include measuring the level of polypeptide concentration for one or more genes that are differentially expressed or present at different levels in allograft recipients undergoing a rejection response versus stable allograft recipients or individuals that have not received an allograft. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a patient data section, including patient medical history (which can include, e.g., age, race, serotype, prior acute cellular rejection episodes, gastroesophageal reflux disease, infection (viral and bacterial), age of transplant recipient, HLA mis-matching, any observed graft dysfunction), as well as administrative patient data such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the monitoring assessment and, if different from the ordering physician, the name of a staff physician who is responsible for the patient's care (e.g., primary care physician).

The report may include a sample data section, which may provide information about the biological sample analyzed in the monitoring assessment, such as the source of biological sample obtained from the patient (e.g. blood, saliva, or type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prediction of the likelihood that the subject will develop a rejection response. The interpretive report can include a diagnosis of a rejection response. The interpretive report can include a characterization of a rejection response. The interpretive report can include, for example, the results of a protein level determination assay (e.g., “1.5 nmol/liter CXCL9 in serum”); and interpretation, i.e. prediction, diagnosis, or characterization. The assessment portion of the report can optionally also include a recommendation(s). For example, where the results indicate that a rejection response is likely, the recommendation can include a recommendation that immunosuppression be provided or enhanced as recommended in the art.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g. prediction, diagnosis or characterization of a rejection response).

As discussed above, the subject methods find use in monitoring an allograft recipient for a rejection response, where by “monitoring an allograft recipient for a rejection response” it is meant predicting the onset of a rejection response, diagnosing the presence or absence of a rejection response, and/or characterizing a rejection response. By “predicting the onset of a rejection response”, it is meant determining the likelihood that an allograft recipient will undergo a rejection response within the next 7 days, e.g. in the next hour, in the next 3 hours, in the next 12 hours, in the next 24 hours, in the next 2 days, in the next 3 days, in the next 4 days, or in the next 7 days. By “diagnosing the presence of a rejection response,” it is meant determining that the allograft patient's immune system has mounted an immune response, e.g. a lymphocytic response, to the allograft. By “characterizing a rejection response” it is meant determining what type of rejection response the allograft recipient is undergoing, e.g. hyperacute, acute, or chronic, and the stage of that rejection response e.g. early, mid-stage, late-stage, and the like, as is well known in the art.

The subject methods further find use in therapeutic applications. In these applications, an allograft recipient is first diagnosed for a rejection response using a protocol described in the preceding section. The subject is then treated using a protocol whose suitability is determined using the results of the monitoring step. For example, where the subject is predicted to have an acute rejection response within the next 7 days or diagnosed to be currently undergoing an acute rejection response, immunosuppressive therapy can be modulated, e.g., increased or altered, as is known in the art for the treatment/prevention of acute rejection. For example, acute rejection may be treated with a short course of high-dose corticosteroids (e.g. Prednisolone, Hydrocortisone), or if this is not enough, a triple therapy regimen comprising a corticosteroid plus a calcineurin inhibitor (e.g. Ciclosporin, Tacrolimus) and an anti-proliferative agent (e.g. Azathioprine, Mycophenolic acid) may be used. Antibodies against specific components of the immune system can be added to this regimen, especially for high-risk patients. mTOR inhibitors (e.g. Sirolimus, Everolimus) can be used in selected patients, where calcineurin inhibitors or steroids are contraindicated. Acute rejection refractory to these treatments may require blood transfusions to remove antibodies against the transplant. In some instances, a bone marrow transplant may be performed where the transplant recipient's immune system is replaced with the donor's immune system, thus enabling the recipient's body to accept the new organ without risk of rejection. Reciprocally, where the subject is predicted to be free of current and near-term acute rejection, the immunosuppressive therapy can be reduced in order to reduce the potential for drug toxicity.

In many embodiments, an allograft recipient is monitored for a rejection response screened once or serially following transplant receipt, e.g., weekly, monthly, bimonthly, half-yearly, yearly, etc., as long as the host is on immunosuppressive therapy. In certain embodiments, monitoring of the host expression profile even after immunosuppressive therapy has been reduced or discontinued is conducted to determine whether the host has maintained the tolerogenic expression profile and may continue for the lifetime of the host.

The subject methods may be employed with a variety of different types of allograft recipients. In many embodiments, the subjects are within the class mammalian, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the animals or hosts, i.e., subjects (also referred to herein as patients) are humans.

The methods may be used to monitor a variety of different types of grafts. Grafts of interest include, but are not limited to: transplanted heart, kidney, lung, liver, pancreas, pancreatic islets, brain tissue, stomach, large intestine, small intestine, cornea, skin, trachea, bone, bone marrow, muscle, bladder or parts thereof.

### Databases of Expression Profiles of Phenotype Determinative Genes

Also provided are databases of expression profiles of rejection responses and non-rejection response. Such databases will typically comprise expression profiles of specific tissues from a transplant recipient that are indicative of one or more of: a near-term AR event (within 3 to 6 months), an ongoing AR response, a previous AR response, a characteristic of an AR response (e.g., steroid resistant/sensitive AR response), a near-term CR event, an ongoing CR response, a previous CR response, a characteristic of a CR response, a stably transplanted recipient undergoing no rejection response, and or any of the aforementioned responses in a transplant recipient receiving an immunosuppressive therapy. Genes that are expressed at one level for one or more of these phenotypes and at a different level for another or more of these phenotypes are phenotype determinative genes.

The expression profiles and databases thereof may be provided in a variety of media to facilitate their use (e.g., in a user-accessible/readable format). “Media” refers to a manufacture that contains the expression profile information of the present invention. The databases of the present invention can be recorded on computer readable media, e.g. any medium that can be read and accessed directly by a user employing a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc. Thus, the subject expression profile databases are accessible by a user, i.e., the database files are saved in a user-readable format (e.g., a computer readable format, where a user controls the computer).

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention, e.g., to and from a user. One format for an output means ranks expression profiles possessing varying degrees of similarity to a reference expression profile. Such presentation provides a skilled artisan (or user) with a ranking of similarities and identifies the degree of similarity contained in the test expression profile to one or more references profile(s).

### Reagents, Systems and Kits

Also provided are reagents, systems and kits thereof for practicing one or more of the above-described methods. The subject reagents, systems and kits thereof may vary greatly. Reagents of interest include reagents specifically designed for use in producing the above-described expression profiles of rejection response phenotype determinative genes from a sample, i.e., a gene expression evaluation element, e.g. a protein level evaluation element made up of one or more reagents useful in determining the level of protein in a sample. The term “system” refers to a collection of reagents, however compiled, e.g., by purchasing the collection of reagents from the same or different sources. The term kit refers to a collection of reagents provided, e.g., sold, together.

One type of such reagent is an array of probe nucleic acids in which the phenotype determinative genes of interest are represented. A variety of different array formats are known in the art, with a wide variety of different probe structures, substrate compositions and attachment technologies (e.g., dot blot arrays, microarrays, etc.). Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

Another type of reagent that is specifically tailored for generating expression profiles of phenotype determinative genes is a collection of gene specific primers that is designed to selectively amplify such genes (e.g., using a PCR-based technique, e.g., real-time RT-PCR). Gene specific primers and methods for using the same are described in U.S. Pat. No. 5,994,076, the disclosure of which is herein incorporated by reference.

Yet another type of reagent that is specifically tailored for generating expression profiles of phenotype determinative genes is a collection of antibodies that bind specifically to the proteins encoded by such genes, e.g. in an ELISA format, in an xMAP™ microsphere format, on a proteomic array, in suspension for analysis by flow cytometry, by western blotting, and immunohistochemistry. Representative antibodies include MAB392 (clone 49106) (R&D Systems), ab17703 (Abcam), ab9720 (Abcam), LS-054341-50 (clone 33D3) (LifeSpan BioSciences); ab54211 (clone 2F7) (Abcam), LS-C47219-100 (clone 2f7b2) (LifeSpan BioSciences); ab2212 (clone MEM-85) (Abcam), and LS-C13446-100 (clone 3H1349) (LifeSpan BioSciences). Methods for using the same are well understood in the art. These antibodies can be provided in solution. Alternatively, they may be provided pre-bound to a solid matrix, for example, the wells of a multi-well dish or the surfaces of xMAP microspheres.

Of particular interest are arrays of probes, collections of primers, or collections of antibodies that include probes, primers or antibodies (also called reagents) that are specific for at least 1 of the genes/proteins listed in Table 1, sometimes a plurality of these genes, e.g., at least 2, 4, 8 or more. In certain embodiments, the collection of probes, primers or antibodies include reagents specific for one or more of CXCL9, PECAM1 and CD44. In certain embodiments, the collection of probes, primers, or antibodies includes reagents specific for all of genes that are from Table 1. The subject probe, primer, or antibody collections may include reagents that are specific only for the genes/proteins that are listed in Table 1, or they may include reagents specific for additional genes/proteins that are not listed in Table 1, such as probes, primers, or antibodies specific for genes/proteins whose expression pattern can be used to evaluate additional transplant characteristics, including but not limited to: immunosuppressive drug toxicity or adverse side effects including drug-induced hypertension; age or body mass index associated genes that correlate with renal pathology or account for differences in recipient age-related graft acceptance; immune tolerance markers in whole blood; genes found in literature surveys with immune modulatory roles that may play a role in transplant outcomes; as well as other methodology-related genes, e.g., for assessing sample quality (extent of nucleic acid or protein degradation), assessing sampling error in biopsy-based studies, and calibrating/normalizing detection levels; and the like. Where the subject probe, primer, or antibody collection includes probes, primers, or antibodies for such additional genes/proteins, in certain embodiments the percent of additional probes, primers, or antibodies that are represented and are not directly or indirectly related to transplantation does not exceed about 50%, usually does not exceed about 25%. In certain embodiments where additional probes, primers, or antibodies are included, a great majority of probes, primers, or antibodies in the collection are transplant characterization probes, primers, or antibodies, where by great majority is meant at least about 75%, usually at least about 80% and sometimes at least about 85, 90, 95% or higher, including embodiments where 100% of the probes, primers, or antibodies in the collection are specific for genes/proteins encoded by phenotype determinative genes.

The systems and kits of the subject invention may include the above-described arrays, gene-specific primer collections, or protein-specific antibody collections. The systems and kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. labeled secondary antibodies, streptavidin-alkaline phosphatase conjugate, chemifluorescent or chemiluminescent substrate, and the like.

The subject systems and kits may also include a phenotype determination element, which element is, in many embodiments, a reference or control expression profile that can be employed, e.g., by a suitable computing means, to make an allograft rejection phenotype determination based on an “input” expression profile, e.g., that has been determined with the above described gene expression evaluation element. Representative phenotype determination elements include databases of expression profiles, e.g., reference or control profiles, as described above.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### Examples

### I. Methods and Materials

### Identification of Differentially Expressed Genes

As described in Dudley, J. T., et al. ((2009) Molecular systems biology 5, 307), we identified microarray experiments in the disease and normal tissues for 280 diseases from Gene Expression Omnibus (GEO) (Barrett, T., et al. (2007) Nucleic Acids Res 35, D760-765), calculated differentially expressed probes with pfp≦5% using RankProd R package (Breitling, R., et al. (2004) FEBS Lett 573, 83-92), and converted the probes IDs to Entrez Gene IDs using AILUN (Chen, R., et al. (2007) Nat Methods 4, 879). For genes with multiple probes, the probes with the most significant pfp values were used. For diseases with multiple data sets, genes differentially expressed in at least one data set were used.

### Prediction of Protein Biomarkers

We have previously constructed a human biofluid proteome database (Dudley, J. T. & Butte, A. J. (2009) Pac Symp Biocomput, 27-38) using data from HUPO Plasma Proteome Project (Omenn, G. S., et al. (2005) Proteomics 5, 3226-3245), a non-redundant list from the Plasma Proteome Institute (Anderson, N. L., et al. (2004) Mol Cell Proteomics 3, 311-326), MAPU Proteome database (Zhang, Y., et al. (2007) Nucleic Acids Res 35, D771-779), and Urinary Exosome database (Pisitkun, T. et al. (2004) Proc Natl Acad Sci USA 101, 13368-13373). We filtered the differentially expressed gene sets with the human biofluid proteome database to predict potential protein biomarkers for each disease.

### Enrichment of Known Protein Biomarkers

We downloaded all protein biomarkers that have biochemical nature, diagnostic application, and Entrez Gene IDs from GVK BIO Online Biomarker Database (GOBIOM). We assigned disease CUIs for clinical indications using Unified Medical Language System (UMLS) (Bodenreider, O. (2004) Nucleic Acids Res 32, D267-270), and compared them with our curated disease CUIs of the microarray data. We found 41 diseases that have both predicted and known protein biomarkers. For each disease, we calculated the association p values between the predicted and known protein biomarkers using Fisher's exact test in R.

### Patients and Samples

We collected 18 acute rejection (AR) and 18 stable (STA) biopsy samples from pediatric renal allograft recipients at Stanford University, and measured gene expression profiles by microarrays. The AR and STA samples were matched for recipient and donor gender, age, donor source, race, time post-transplant, HLA matches, and they are all under the same either double (Tacrolimus and MMF) or triple immunosuppression (Tacrolimus, MMF and steroid) protocol with the same Daclizmab induction (Sarwal, M. M., et al. (2001) Transplantation 72, 13-21). The mean±standard deviation data for demographic and clinical variables for the patients are provided in Table 1. All 36 samples were used to categorize the sample as AR with biopsy proven according to the Banff classification (Tusher, V. G., (2001) Proc Natl Acad Sci USA 98, 5116-5121) on tubulitis, interstitial inflammation, glomerulitis, and vasculitis (n=18, Banff grade samples were IA, IB, and IIA not including boarder line), or as STA (n=18) if there was absence of AR and any other substantive pathology and with stable graft function on protocol biopsy, which we conducted at 3, 6, 12, and 24 months after transplantation and for graft dysfunction (Sarwal, M. M., et al. (2001) Transplantation 72, 13-21; Sarwal, M. M., et al. (2003) Transplantation 76, 1331-1339). None of patients have BKV infection. All pathology analyses were performed by a single blinded pathologist (NK) at Stanford University. Written informed consent was obtained from all the subjects. The study was approved by the Stanford University Institutional Review Board.

TABLE 1
Patient demographics of AR versus STA allograft biopsies in pediatric
renal transplant microarray study.

| | | | |
|---|---|---|---|
| Clinical Characteristics | AR (n = 18) | STA (n = 18) | P value |

Recipients

| | | | |
|---|---|---|---|
| Gender (% females) | 39% | 44% | 0.74 |
| Mean age (year) | 11.9 ± 6.1 | 11.7 ± 5.5 | 0.92 |
| Age range (year) | 1-21 | 1-19 | |
| Immunosuppression, | 61% | 33% | 0.1 |
| (% SF)^{#} | | | |
| Sample collection time^{$} | 17.4 ± 25.2 | 9.3 ± 6.1 | 0.2 |
| (month, post-transplant) | | | |
| Sample collection time | 1-97 | 2-25 | |
| range (month) | | | |
| Race (1, 2, 3, 4, 5)* | 73%, 18%, 0%, | 35%, 12%, 12%, | 0.18 |
| | 0%, 25% | 24%, 17% | |
| ESRD (1, 2, 3, 4, 5, 6)** | 17%, 8%, 17%, | 17%, 12%, 18%, | 0.06 |
| | 41%, 8%, 8% | 0%, 6%, 47% | |

Donors

| | | | |
|---|---|---|---|
| Source (% LRD) | 50% | 59% | 0.6 |
| Gender (% females) | 43% | 35% | 0.67 |
| Age (year) | 27.1 ± 13.6 | 29.7 ± 9.7 | 0.57 |
| Age range (year) | 4-44 | 15-55 | |
| HLA match | 2.2 ± 1.8 | 1.2 ± 1.3 | 0.2 |

Values are mean ± SD (Standard Deviation).
AR: Acute Rejection;
STA: stable;
SF: Steroid-free drug treatment;
ESRD: End stage renal disease;
LRD: Living related donor.
% SF (#) is the percentage of patients with steroid-free drug treatments.
Race (*) is defined as follows: 1 = Caucasian; 2 = Hispanic; 3 = Asian; 4 = African American; 5 = Other.
ESRD (**) is defined by the following categories: 1 = Glomerulonephritis; 2 = Polycystic Kidney Disease; 3 = Dysplasia; 4 = Reflux Nephropathy; 5 = Obstructive Uropathy; 6 = Other.
The difference in the sample collection time ($) between AR and STA was caused by two AR sample collected at 69 and 97 months after transplant. The remaining 16 AR samples were collected at 9 ± 7 months after transplants, which is the same as that of stable patients. Removing these two late-stage AR samples only resulted in minor changes in the AR signature.

For ELISA experiments in renal transplant, we collected serum samples from 19 AR and 20 STA patients without BKV infection. All serum samples were obtained within 24 hours of a clinically indicated or protocol graft biopsy, and each sample was matched with the patient's biopsy. AR samples were biopsy proven according to the Bank classification (IA, IB, IIA, IIB, not including boarder line). We also collected plasma samples from 32 AR, 31 STA patients without CMV infection after cardiac transplant at Stanford Hospital. To minimize the loss in the sample processing, plasma were directly used in the ELISA study. Acquisition of samples in both studies was approved by the Stanford University Institutional Review Board. All AR samples have ISHLT grade of 3A or 3B. Stable samples showed an absence of AR and any other substantive pathology.

### Microarray Experiments

The total RNA used for the first-strand cDNA synthesis using a T7 promoter-linked oligo(dT) primer following the standard protocol for the Affymetrix One-Cycle cDNA Synthesis Kit (Affymetrix, Part. 900493). After second strand cDNA synthesis, biotin-labeled cRNA was prepared in an in vitro transcription reaction using the GeneChip IVT Labeling Kit (Affymetrix). Ten microgram of fragmented cRNA was used for hybridization on the Affymetrix Human Genome U133 Plus 2.0 microarrays according to the manufacturer's instructions. The raw and processed data have been deposited into GEO with an accession ID of GSE14328.

### Microarray Data Analysis

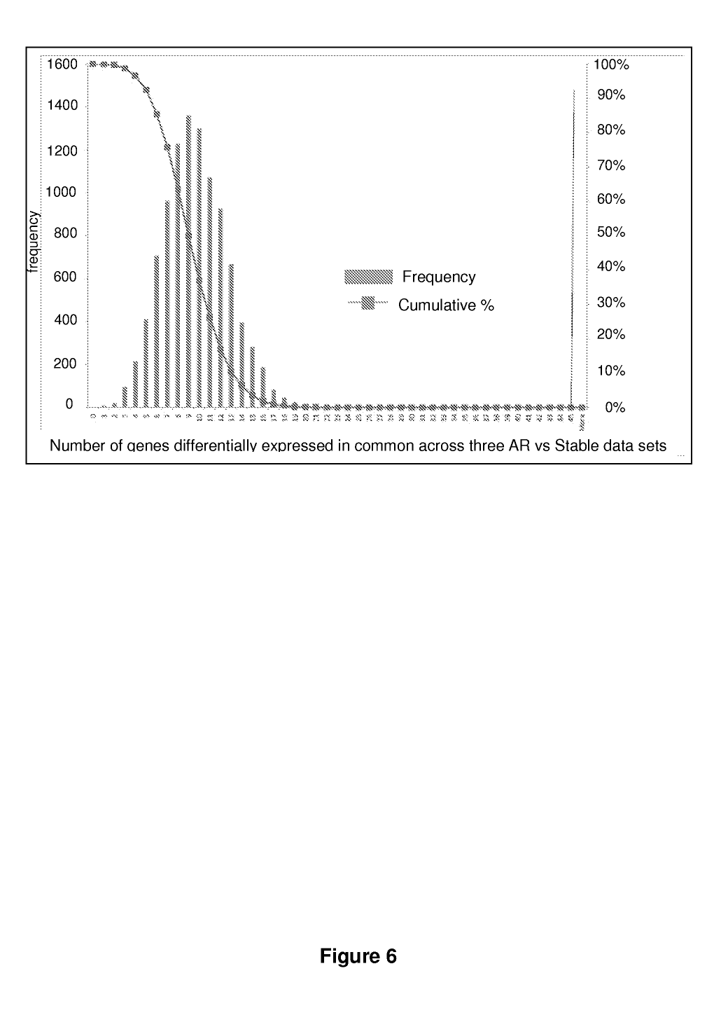

All three data sets (pediatric renal, adult renal, and adult heart) were normalized by the quantile-quantile method using dChip software. Probes significantly upregulated in AR versus STA were identified using Significant Analysis of Microarray (SAM (Tusher, V. G., (2001) Proc Natl Acad Sci USA 98, 5116-5121); q≦0.05). All significant AR probes were related to Entrez Gene IDs using AILUN (Chen, R., et al. (2007) Nat Methods 4, 879). We limited AR genes as significantly upregulated in AR compared to STA. We found 9,086 pediatric renal AR genes, 2316 adult renal AR genes, and 283 heart AR genes (FIG. 6).

The number of heart AR genes was significantly less than those of kidney AR genes due to different platforms and organs. Publicly available heart AR data were measured on a 70mer spotted array from NIH/NIAID (GEO record numbers GPL1053 & GSE4470). It contains 8972 probes related to 8437 Entrez Gene IDs, and was smaller than our Affymetrix U133 plus 2.0 array used for the pediatric renal study and the Affymetrix U95 array used for the publicly available adult renal study (GEO record numbers GPL91, GDS724).

To make the number of AR genes comparable between pediatric and adult renal studies, we added an extra filter of fold ≧2 on the pediatric renal study. We obtained 2,805 pediatric renal AR genes, 2,316 adult renal AR genes, and 283 heart AR genes (FIG. 2). Intersecting these three AR gene lists, we found 45 common upregulated AR genes irrespective of transplanted organs.

### ELISA Validation

Ten proteins in serum were measured by using commercial ELISA kits. ELISA kits for PECAM1 (Cat. No. ab45910), CD44 (Cat. No. ab45912), and SELL (Cat. No. ab45917) were purchased from ABCam Inc (Cambridge, Mass.); an ELISA kit for SA100A4 (Cat. No. CY-8059) was purchased from MBL International (Woburn, Mass.); ELISA kits for CCL4 (Cat. No. DMB00), CXCL11 (cat. No. DCX110) and CXCL9 (cat. No. DCX900) were purchased from R&D Systems (Minneapolis, Minn.); an ELISA kit for STAT-1 (cat. CBA034) was purchased from Calbiochem (Gibbstown, N.J.); an ELISA Kit for BIRC5/Survivin (Cat. No. 900-111) was purchased from Assay Designs (Ann Arbor, Mich.); and an ELISA assay for CCL8 was developed using the DuoSet ELISA Development System for human CCL8/MCP-2 from R&D Systems (Cat. No. DY281).

Sample, reagent, and buffer preparation was done according to the manufacturer manuals and the assay was performed by following manual instructions exactly. Microwell plates were read by a SPECTRAMax 190 microplate reader (Molecular Devices, Sunnyvale, Calif.). Protein concentrations were determined from a standard curve generated from standards supplied with the kits. Similarly, the protein concentrations of PECAM1, CXCL9 and CD44 in the plasma samples of heart transplant patients were measured by ELISA kits.

### Immunohistochemistry

Immunohistochemical staining was performed on 4 μm sections obtained from formalin fixed paraffin embedded tissues using mouse monoclonal anti-human antibodies directed against PECAM-1 (DAKO, Carpinteria, Calif.; Catalog #M823; dilution 1:150). Heat induced antigen retrieval was performed with Ventana Benchmark Autostainer. The staining was optimized using appropriate positive and negative controls.

### Statistical Analysis

T test and chi-square tests were used for continuous and categorical clinical variables comparison in patient demographics using SAS 9.1.3 (SAS Institute Inc., Cary, N.C.). Protein concentrations from ELISAs were compared between AR and STA using Mann-Whitney U test in R. P-values≦0.05 were considered statistically significant. The enrichment of known protein biomarkers in differentially expressed genes were calculated using Fisher's exact test in R.

### II. Results

We identified a set of proteins that were differentially expressed at the mRNA levels between disease and normal tissues for 41 diseases, with detectable protein abundance in the serum or urine at normal conditions. Using a previously described pipeline (Dudley, J. T., et al. (2009) Molecular systems biology 5, 307), we identified gene expression data in disease and control tissue samples in GEO, and calculated a set of differentially expressed genes with a percentage of false prediction (pfp)≦5% for each disease using RankProd R package. For diseases with multiple data sets, we included genes that were differentially expressed in at least one of the data sets. We then filtered the gene sets with a biofluid proteome database, a database previously constructed (Dudley, J. T. & Butte, A. J. (2009) Pac Symp Biocomput, 27-38) that contains 3,638 proteins with detectable abundance in serum, plasma, or urine, to report candidate protein biomarkers that can be detected in the serum or urine.

We then compared our predicted protein sets with known diagnostic protein biomarkers in the clinical and preclinical studies in the GVK BIO Online Biomarker Database (GOBIOM), collected from global clinical trials. GOBIOM contains 6,098 known biomarkers for 368 therapeutic indications with 23,166 unique references. For 22 out of 41 diseases, known diagnostic protein biomarkers were statistically significantly enriched in our predicted protein sets (p<0.05, Fisher's exact, Table 2). For 14 out of the remaining 18 diseases, we only had a single gene expression data set.

TABLE 2
Clinically and pre-clinically validated diagnostic protein biomarkers are significantly
enriched in differentially expressed genes for 22 out of 41 diseases.

| | | Predicted | Known | | |
|---|---|---|---|---|---|
| | Microarray | protein | Protein | True | |
| Disease | Data | Biomarkers* | Biomarkers** | Positive *** | P value^{$} |
| Breast Cancer | GSE53, | 1845 | 134 | 63 | 2.26 × 10⁻²⁴ |
| | GSE1378, | | | | |
| | GSE1379, | | | | |
| | GSE1872, | | | | |
| | GSE2155, | | | | |
| | GSE2429, | | | | |
| | GSE2528, | | | | |
| | GSE3744, | | | | |
| | GSE4382 | | | | |
| Lung Cancer, | GSE1037 | 1064 | 44 | 23 | 2.21 × 10⁻¹¹ |
| Non-Small Cell | | | | | |
| Diabetes Mellitus, | GSE710, | 439 | 17 | 9 | 5.30 × 10⁻⁹ |
| Type 2 | GSE642, | | | | |
| | GSE2470, | | | | |
| | GSE3068, | | | | |
| | GSE6428 | | | | |
| Chronic Obstructive | GSE475, | 217 | 18 | 5 | 5.14 × 10⁻⁶ |
| Pulmonary Disease | GSE1650, | | | | |
| | GSE3320, | | | | |
| | GSE10964 | | | | |
| Melanoma | GSE3189, | 1005 | 49 | 13 | 3.91 × 10⁻⁵ |
| | GSE4587 | | | | |
| Alzheimer's Disease | GSE1297, | 1414 | 19 | 8 | 8.04 × 10⁻⁵ |
| | GSE5281 | | | | |
| | (3 data sets) | | | | |
| Crohn's Disease | GSE1710, | 1515 | 9 | 6 | 1.94 × 10⁻⁴ |
| | GSE3365, | | | | |
| | GSE6731 | | | | |
| Cystic Fibrosis | GSE765, | 234 | 8 | 3 | 4.56 × 10⁻⁴ |
| | GSE769, | | | | |
| | GSE3100 | | | | |
| Hypercholesterolemia | GSE3889 | 712 | 3 | 3 | 4.81 × 10⁻⁴ |
| Wilms tumor | GSE2712 | 192 | 2 | 2 | 6.53 × 10⁻⁴ |
| Sickle Cell Anaemia | GSE9877 | 1437 | 7 | 5 | 1.15 × 10⁻³ |
| Myelodysplastic | GSE2779, | 779 | 10 | 4 | 1.88 × 10⁻³ |
| Syndromes | GSE4619 | | | | |
| Leukemia, Chronic | GSE2466 | 671 | 21 | 7 | 2.90 × 10⁻³ |
| Lymphocytic | | | | | |
| Lung Cancer, | GSE1037 | 986 | 4 | 3 | 4.44 × 10⁻³ |
| Small Cell | | | | | |
| HIV Infection | GSE2171, | 367 | 24 | 4 | 7.34 × 10⁻³ |
| | GSE2504, | | | | |
| | GSE6740 | | | | |
| Prostate Cancer | GSE1413, | 302 | 88 | 6 | 0.012 |
| | GSE3868 | | | | |
| Diabetes Mellitus, | GSE710, | 214 | 9 | 2 | 0.018 |
| Type 1 | GSE1623, | | | | |
| | GSE1659, | | | | |
| | GSE2254, | | | | |
| | GSE4616 | | | | |
| Lymphoma | GSE60, | 28 | 27 | 2 | 0.023 |
| | GSE3211 | | | | |
| Transitional Cell | GSE3167 | 899 | 3 | 2 | 0.025 |
| Carcinoma | | | | | |
| Liver Cirrhosis | GSE1843, | 905 | 4 | 2 | 0.028 |
| | GSE6764 | | | | |
| Ulcerative Colitis | GSE1710, | 1301 | 7 | 3 | 0.030 |
| | GSE3365, | | | | |
| | GSE6731 | | | | |
| Heart Failure | GSE1988 | 96 | 2 | 1 | 0.044 |
| Colon Cancer | GSE2178, | 451 | 17 | 2 | 0.096 |
| | GSE4107 | | | | |
| Rheumatoid Arthritis | GSE1919, | 307 | 15 | 2 | 0.098 |
| | GSE2053, | | | | |
| | GSE3592 | | | | |
| Cardiomyopathy | GSE1869, | 1172 | 5 | 2 | 0.11 |
| | GSE5406 | | | | |
| Thyroid Cancer | GSE5364 | 933 | 6 | 2 | 0.12 |
| Obesity | GSE474, | 161 | 10 | 1 | 0.13 |
| | GSE4692, | | | | |
| | GSE4697 | | | | |
| Atherosclerosis | GSE363 | 25 | 21 | 1 | 0.15 |
| Sarcoidosis | GSE1907 | 369 | 7 | 1 | 0.51 |
| Hypertension | GSE1674 | 10 | 11 | 0 | 1 |
| Vitamin B12 Deficiency | GSE2779 | 3 | 2 | 0 | 1 |
| Testicular Cancer | GSE1818 | 100 | 14 | 0 | 1 |
| Bipolar Disorder | GSE5389 | 267 | 1 | 0 | 1 |
| Schizophrenia | GSE4036 | 116 | 5 | 0 | 1 |
| Leukemia, Acute | GSE2191 | 148 | 11 | 0 | 1 |
| Myeloid | | | | | |
| Parkinson's Disease | GSE7621 | 100 | 6 | 0 | 1 |
| Thymic Carcinoma | GSE2501 | 65 | 6 | 0 | 1 |
| Obstructive Sleep | GSE1873 | 34 | 3 | 0 | 1 |
| Apnea | | | | | |
| Osteoarthritis | GSE1919 | 60 | 3 | 0 | 1 |
| Inflammatory Bowel | GSE4183 | 619 | 2 | 0 | 1 |
| Disease | | | | | |
| Multiple Sclerosis | GSE10064 | 11 | 3 | 0 | 1 |

The numbers listed under “Microarray data” are Gene Expression Omnibus (GEO) record IDs for the gene expression data in disease vs. normal tissues.
Predicted protein biomarkers (*) are the number of genes that were differentially expressed in any one of the disease tissues at the mRNA level (fpf ≦ 0.05, RankProd R package) with detectable protein abundance in the biofluid proteome database (see Methods).
Known protein biomarkers (**) are the number of known diagnostic protein biomarkers in clinical and preclinical studies from GOBIOM (GVK BIO Online Biomarker Database, http://www.gobiomdb.com/gobiom/).
True positives (***) are the number of correctly predicted diagnostic protein biomarkers.
The p values ($) were calculated to evaluate whether known protein biomarkers were significantly enriched in our predicted gene set using Fisher's exact test.

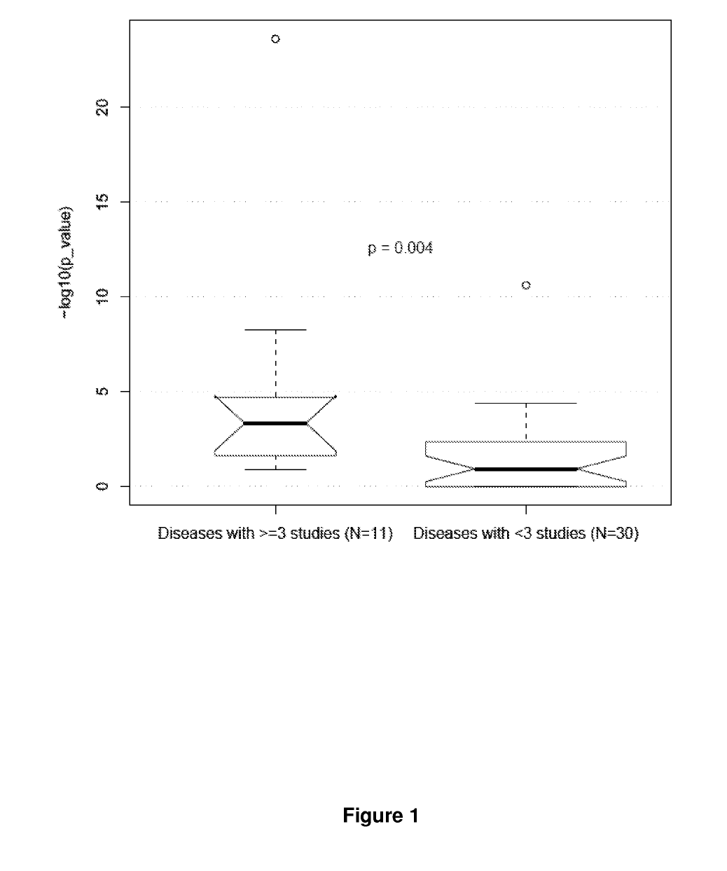

In 82% (9 of 11) of diseases with 3 or more data sets analyzed, known diagnostic protein biomarkers were significantly enriched in our predicted protein sets. The −log 10 (p-value) in diseases with three or more data sets (N=11) is statistically significantly higher than those in diseases with less than three data sets (N=30) (p=0.004, Fisher's exact, FIG. 1). Therefore, the more gene expression data sets that are available for a disease, the more likely known serum/urine protein biomarkers are going to be significantly differentially expressed across any one of those data sets, suggesting the likelihood of finding new biomarkers increases with more available data sets.

We applied our method on transplant rejection to search for serum-detectable protein biomarkers for the diagnosis of acute rejection (AR). We integrated biopsy-based gene expression microarray studies from pediatric renal, adult renal (Flechner, S. M., et al. (2004) Am J Transplant 4, 1475-1489), and adult cardiac (Morgun, A., et al. (2006) Circ Res 98, e74-83) transplantation, identified genes commonly upregulated in AR compared to stable graft function, then measured their protein abundance in serum to identify cross-organ AR protein biomarkers (FIG. 2). The first of the three studies was performed in pediatric renal transplantation, and compared gene expression profiles in the biopsy samples from 18 AR patients and 18 patients with stable graft function (STA) at the absence of AR and any other substantive pathology. We found 2,805 genes to have increased expression in biopsies with AR compared to STA (q≦0.05, fold≧2, SAM (Tusher, V. G., (2001) Proc Natl Acad Sci USA 98, 5116-5121)).

To find a cross-organ AR signature, we combined the data from this study with data from two other transplant studies, retrieved from the NCBI Gene Expression Omnibus (GEO) (Barrett, T., et al. (2007) Nucleic Acids Res 35, D760-765). One study compared 13 AR to 19 STA biopsy samples after adult kidney transplant (GEO record number GDS724 (Flechner, S. M., et al. (2004) Am J Transplant 4, 1475-1489)), yielding 2,316 upregulated AR genes (q≦0.05, SAM). The second study compared 12 AR to 13 non-rejection biopsy samples after cardiac transplant (GEO record number GSE4470 (Morgun, A., et al. (2006) Circ Res 98, e74-83)), yielding 283 upregulated AR genes (q≦0.05, SAM). By intersecting the three data sets, we identified a gene expression signature for AR containing 45 genes in common, irrespective of the specific studies or transplanted organs (Table 3).

TABLE 3
Forty-five AR genes commonly upregulated among solid-
organ transplant biopsy gene expression studies

| | | Pediatric | | Adult | | | | Human | Tested | |
|---|---|---|---|---|---|---|---|---|---|---|
| | Reference | Kidney AR | | kidney AR | | Heart AR | | Proteome | by | AR |
| Gene | Sequence | Fold | q | Fold | q | Fold | q | Database | ELISA | Marker |
| CXCL9 | NM_002416 | 4.0 | 0 | 3.1 | 0.001 | 14.7 | 0 | | Y | Y |
| CXCL11 | NM_005409 | 4.9 | 0 | 1.7 | 0.02 | 6.3 | 0.005 | | Y | |
| CXCR4 | NM_001008540 | 8.7 | 0 | 1.5 | 0.02 | 2.4 | 0.009 | | | |
| STAT1 | NM_007315 | 7.0 | 0 | 2.3 | 0 | 3.0 | 0 | Plasma | Y | |
| CCL4 | NM_002984 | 3.5 | 0 | 4.2 | 0.001 | 3.0 | 0.03 | Plasma | Y | |
| C6orf32 | NM_014722 | 4.4 | 0 | 4.2 | 0.003 | 1.8 | 0.005 | | | |
| | NM_015864 | | | | | | | | | |
| MARCKS | NM_002356 | 6.5 | 0 | 1.4 | 0.04 | 2.3 | 0 | Urine, | | |
| | | | | | | | | Plasma | | |
| IGSF6 | NM_005849 | 4.3 | 0 | 2.3 | 0.02 | 3.2 | 0 | | | |
| CD2 | NM_001767 | 2.1 | 0.006 | 1.3 | 5E−4 | 6.2 | 0 | | | |
| TRPM1 | NM_002420 | 6.2 | 7E−5 | 1.1 | 0.04 | 1.7 | 0.03 | Plasma | | |
| IL10RA | NM_001558 | 5.3 | 0 | 1.1 | 0.03 | 1.8 | 0.03 | | | |
| RARRES3 | NM_004585 | 2.7 | 0 | 1.4 | 0.003 | 4.0 | 0 | | | |
| NR4A2 | NM_006186 | 5.3 | 0 | 1.3 | 0.02 | 1.4 | 0.04 | | | |
| PTPRC | NM_002838 | 2.5 | 0 | 2.2 | 0.0009 | 3.2 | 0 | | | |
| LEF1 | NM_001130713 | 2.7 | 2E−4 | 1.7 | 0.01 | 3.2 | 0 | | | |
| | NM_001130714 | | | | | | | | | |
| | NM_016269 | | | | | | | | | |
| TAP1 | NM_000593 | 2.7 | 0 | 1.4 | 0.002 | 3.3 | 0 | | | |
| CTSS | NM_004079 | 3.5 | 0 | 1.6 | 0.01 | 2.3 | 0.005 | Plasma | | |
| ISG20 | NM_002201 | 2.4 | 4E−4 | 1.3 | 0.03 | 3.6 | 0 | | | |
| CCL8 | NM_005623 | 3.9 | 7E−5 | 1.1 | 0.03 | 2.1 | 0.03 | | Y | |
| BASP1 | NM_006317 | 2.8 | 7E−5 | 2.0 | 0.01 | 2.0 | 0.04 | Urine, | | |
| | | | | | | | | Plasma | | |
| SLC2A3 | NM_006931 | 2.8 | 0 | 2.0 | 0.01 | 1.8 | 0 | | | |
| LCP2 | NM_005565 | 2.5 | 0 | 2.3 | 0.003 | 1.6 | 0.03 | | | |
| HLA-DMA | NM_006120 | 2.3 | 0.001 | 1.4 | 0.003 | 2.7 | 0.005 | | | |
| BIRC5 | NM_001012270 | 3.1 | 0 | 1.1 | 0.02 | 2.2 | 0.02 | | Y | |
| | NM_001012271 | | | | | | | | | |
| | NM_001168 | | | | | | | | | |
| HLA-DMB | NM_002118 | 2.4 | 2E−4 | 1.4 | 0.001 | 2.4 | 0 | | | |
| CASP4 | NM_001225 | 2.1 | 4E−4 | 1.8 | 0.001 | 2.3 | 0 | | | |
| | NM_033306 | | | | | | | | | |
| SELL | NM_000655 | 2.0 | 0.003 | 1.3 | 0.04 | 2.9 | 0 | Plasma | Y | |
| HLA-F | NM_001098478 | 2.2 | 0 | 1.3 | 0.02 | 2.6 | 0 | | | |
| | NM_001098479 | | | | | | | | | |
| | NM_018950 | | | | | | | | | |
| CD44 | NM_000610 | 3.5 | 0 | 1.1 | 0.03 | 1.5 | 0.02 | Urine, | Y | Y |
| | NM_001001389 | | | | | | | Plasma | | |
| | NM_001001390 | | | | | | | | | |
| | NM_001001391 | | | | | | | | | |
| | NM_001001392 | | | | | | | | | |
| HLA-DQB1 | NM_002123 | 2.6 | 0 | 1.5 | 0.01 | 2.1 | 0.02 | | | |
| PIK3CD | NM_005026 | 2.1 | 0 | 1.1 | 0.03 | 2.7 | 0 | | | |
| SH2D2A | NM_003975 | 2.3 | 0 | 1.1 | 0.02 | 2.4 | 0.005 | | | |
| CCNB2 | NM_004701 | 2.4 | 5E−4 | 1.2 | 0.002 | 2.1 | 0.005 | Plasma | | |
| HLA-DRA | NM_019111 | 2.0 | 0.01 | 1.3 | 9E−4 | 2.3 | 0.005 | | | |
| B2M | NM_004048 | 2.2 | 7E−5 | 1.2 | 0.01 | 2.2 | 0.005 | Urine | | |
| DIAPH1 | NM_001079812 | 2.8 | 0.007 | 1.1 | 0.03 | 1.7 | 0.009 | Plasma | | |
| | NM_005219 | | | | | | | | | |
| USP34 | NM_014709 | 2.1 | 0 | 1.8 | 0.001 | 1.7 | 0.03 | Plasma | | |
| SCAND2 | NR_003654 | 2.7 | 7E−4 | 1.3 | 5E−4 | 1.6 | 0.02 | | | |
| | NR_004859 | | | | | | | | | |
| RUNX1 | NM_001001890 | 2.2 | 0 | 1.2 | 0.003 | 2.1 | 0.03 | | | |
| | NM_001122607 | | | | | | | | | |
| | NM_001754 | | | | | | | | | |
| S100A4 | NM_002961 | 2.6 | 7E−5 | 1.1 | 0.02 | 1.6 | 0.03 | Urine | Y | |
| | NM_019554 | | | | | | | | | |
| PECAM1 | NM_000442 | 2.4 | 0 | 1.3 | 9E−4 | 1.5 | 0.02 | Urine, | Y | Y |
| | | | | | | | | Plasma | | |
| MDK | NM_001012333 | 2.1 | 5E−4 | 1.1 | 0.02 | 1.8 | 0.005 | Plasma | | |
| | NM_001012334 | | | | | | | | | |
| | NM_002391 | | | | | | | | | |
| MELK | NM_014791 | 2.0 | 4E−4 | 1.2 | 0.003 | 1.7 | 0.02 | | | |
| CDKN3 | NM_001130851 | 2.3 | 0 | 1.1 | 0.02 | 1.4 | 0.005 | | | |
| | NM_005192 | | | | | | | | | |
| CPD | NM_001304 | 2.0 | 0.004 | 1.2 | 0.02 | 1.4 | 0.04 | Plasma | | |

To evaluate the significance of getting 45 genes in common, we shuffled the gene labels in the three data sets and repeated the analysis 100,000 times. The numbers of intersecting genes showed a normal distribution around N=9 (FIG. 6). This result suggested that the probability of finding 17 or more common AR genes by random is less than 1%, and the probability of finding 24 or more AR genes by random is less than 1×10-5.

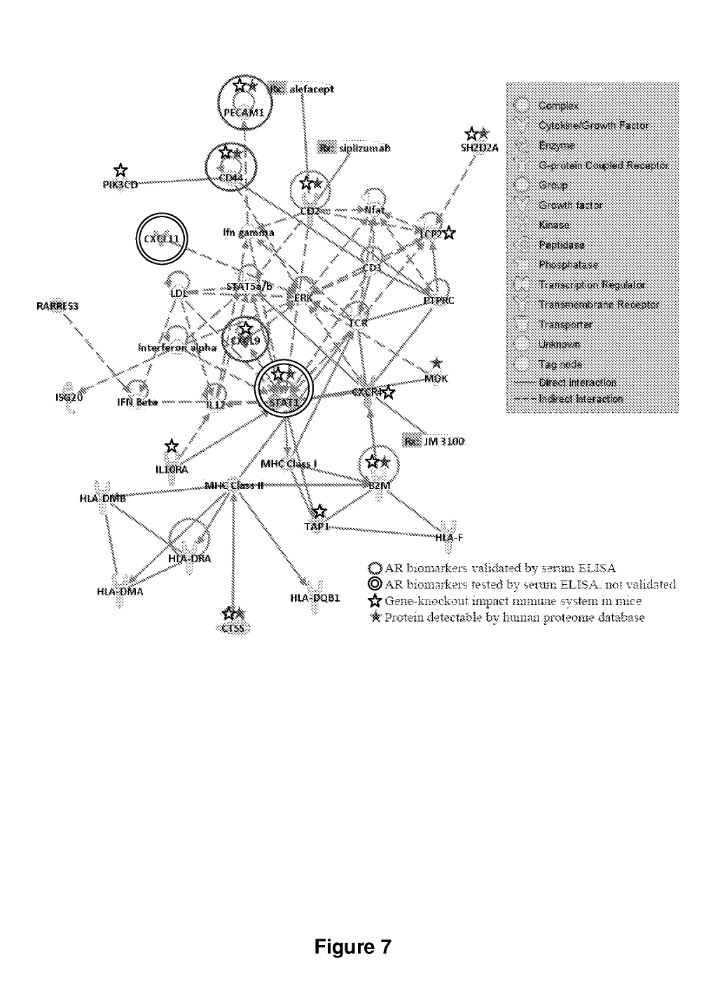

We then analyzed the functions of these 45 cross-organ AR genes using Ingenuity Pathway Analysis (IPA, Ingenuity Systems), and SymAtlas (Su, A. I., et al. (2004) Proc Natl Acad Sci USA 101, 6062-6067). As expected, 28 AR genes were involved in inflammatory response (p=3.37×10-17, Fisher's exact; p<3.56×10-3 after Benjamini-Hochberg multi-test correction). Furthermore, 23 AR genes were involved in cell-mediated immune response (p=3.34×10-15; p<2.97×10-3, Benjamini-Hochberg correction). For each AR gene, we retrieved its mRNA expression in 74 tissue/cell types from SymAtlas, and identified the cell type that expressed that gene most highly. We found that cross-organ AR genes were highly expressed in CD14+ monocytes (p=0.003, Fisher's exact)—seven AR genes have highest expression in CD14+ monocytes, including CD44, IL10RA, S100A4, IGSF6, CTSS, CASP4, and SCAND2-suggesting an important role of monocyte-activated pro-inflammation in transplant rejection, which could be used for the early diagnosis of AR. Additionally, 23 out of the 45 AR genes were involved in a single pathway associated with inflammatory response, antimicrobial response and cellular movement regulated by STAT-1 (FIG. 7), suggesting the STAT-1-regulated pro-inflammatory pathway might be the common mechanism for allograft acute rejection irrespective of transplanted organs. Consistent with this, it has recently been shown that a single treatment of a decoy oligodeoxynucleotide neutralizing STAT-1 effectively reduced cellular rejection in mouse heart allograft through an early decline in pro-inflammatory gene expression and a later drop in mononuclear cell infiltration (Stojanovic, T., et al. (2009) Basic Res Cardiol 104, 719-729).

ELISA kits were readily available for ten of the 45 candidate proteins. We used the kits to measure all ten proteins in serum samples collected within 24 hours from 19 patients with biopsy-proven AR and 20 patients with absence of AR and any other substantive pathology (STA) in a pediatric and young adult renal transplant study. None of the patients had BK virus infection. No samples used in the ELISA study were from patients in the microarray study. The AR/STA samples were matched for recipient and donor gender, age, type of immunosuppression, time post-transplant, race, and type of end stage renal diseases (Table 4).

TABLE 4
Patient demographics of renal transplant in ELISA study.

| | | | |
|---|---|---|---|
| Clinical Characteristics | AR (n = 19) | STA (n = 20) | P value |

Recipients

| | | | |
|---|---|---|---|
| Gender (% females) | 53% | 65% | 0.43 |
| Mean age (year) | 11.5 ± 6.5 | 13.6 ± 4.1 | 0.24 |
| Age range (year) | 1.5-18.7 | 2.4-19.0 | |
| Immunosuppression | 32% | 50% | 0.24 |
| (% SF)^{#} | | | |
| Sample collection time | 8.4 ± 6.7 | 6.2 ± 1.4 | 0.17 |
| (month, post-transplant) | | | |
| Sample collection time | 0.1-65.0 | 5.4-12.0 | |
| range (month) | | | |
| Race (1, 2, 3, 4, 5)* | 37%, 0%, 0%, | 60%, 0%, 0%, | 0.32 |
| | 47%, 16% | 25%, 15% | |
| ESRD (1, 2, 3, 4, 5, 6)** | 21%, 0%, 11%, 5%, | 11%, 5%, 5%, | 0.81 |
| | 5%, 58% | 16%, 5%, 58% | |

Donors

| | | | |
|---|---|---|---|
| Source (% LRD) | 32% | 55% | 0.14 |
| Gender (% females) | 37% | 45% | 0.6 |
| Age (year) | 26.7 ± 9.4 | 30.7 ± 11.6 | 0.24 |
| Age range (year) | 5-42 | 17-54 | |
| HLA match | 1.0 ± 1.3 | 1.9 ± 1.3 | 0.1 |

Values are mean ± SD (Standard Deviation).
AR: Acute Rejection;
STA: stable; SF: Steroid-free drug treatment;
ESRD: End stage renal disease.
LRD: Living related donor.
% SF (#) is the percentage of patients with steroid-free drug treatments.
Race (*) is defined as: 1 = Caucasian; 2 =Hispanic; 3 = Asian; 4 = African American; 5 = Other.
ESRD (**) categories are: 1 = Glomerulonephritis; 2 = Polycystic Kidney Disease; 3 = Dysplasia; 4 = Reflux Nephropathy; 5 = Obstructive Uropathy; 6 = Other.

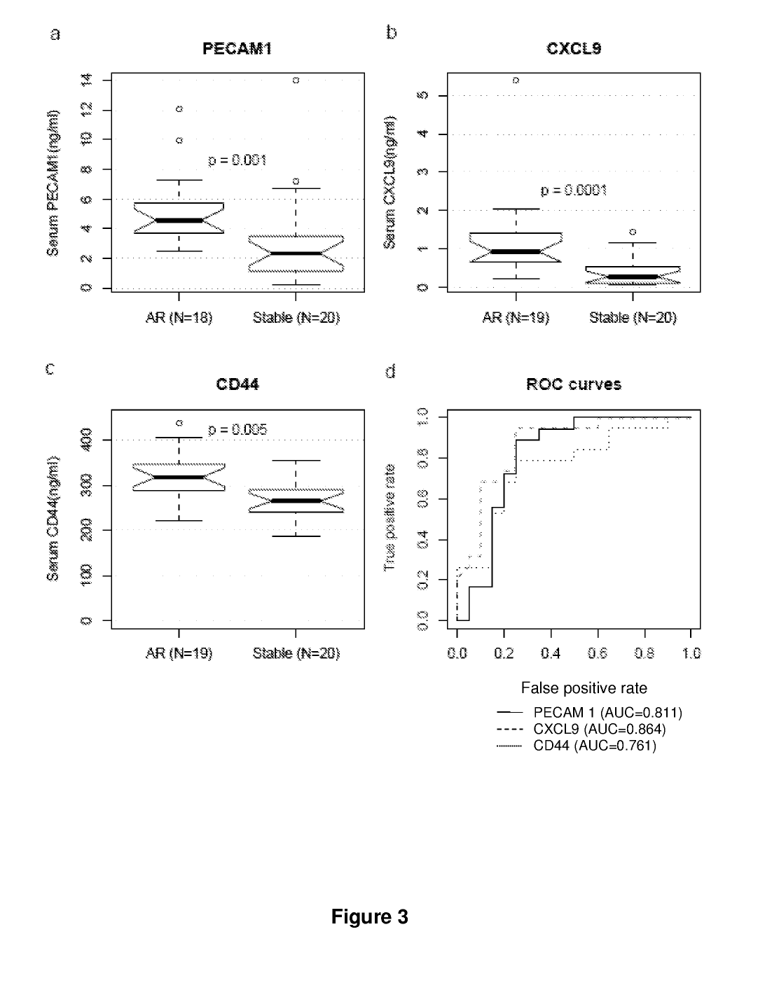

Three of ten proteins were statistically significantly upregulated in the AR serum samples compared to the STA samples after renal transplantation (FIG. 3). They are PECAM1 (CD31 antigen or platelet/endothelial cell adhesion molecule), CXCL9 (MIG, chemokine ligand 9), and CD44 (hyaluronic acid receptor) with Mann-Whiney U test p-values of 1×10-3, 1×10-4, and 5×10-3, respectively. Receiver Operating Characteristics (ROC) curves showed the ability of each individual protein to distinguish AR from STA (FIG. 3d). The areas under the ROC curves (AUC) were 0.811, 0.864, and 0.761 for PECAM1, CXCL9, and CD44, respectively. At optimal performance, PECAM1 distinguished AR from STA with 89% sensitivity and 75% specificity; CXCL9: 78% sensitivity and 80% specificity; CD44: 80% sensitivity and 75% specificity.

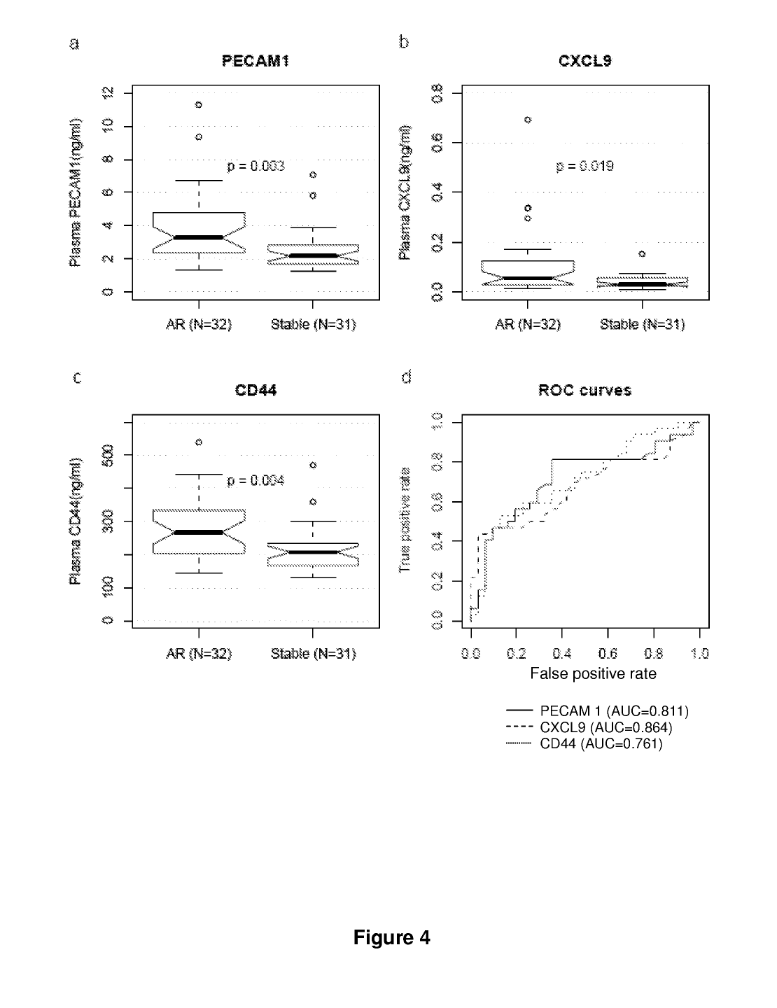

We then measured the concentration of these three proteins in the plasma samples of cardiac allograft recipients to identify cross-organ AR biomarkers. We compared 32 patients with AR and 31 patients with STA. Demographics were matched between AR and STA samples (Table 5). None of the patients had CMV infection. Interestingly, all three markers showed significant upregulation in AR compared to STA samples, with Mann-Whitney U test p values of 3×10-3 (PECAM1), 0.019 (CXCL9), and 4×10-3 (CD44) (FIG. 4). The areas under the ROC curves were 0.716, 0.672, and 0.711 for PECAM1, CXCL9, and CD44 to distinguish AR from STA, respectively. Although the protein abundance of CXCL9 in urine (Hauser, I. A., et al (2005) J Am Soc Nephrol 16, 1849-1858) and of soluble CD44 in plasma (Rouschop, K. M., et al. (2006) Kidney Int 70, 1127-1134) has been shown to increase in renal AR compared with STA, and PECAM1 on the surface of macrophages has been shown to distinguish lung transplant rejection (Rizzo, M., et al. (2000) J Heart Lung Transplant 19, 858-865) and to be involved (not diagnostic) in mouse models of cardiac transplant rejection (Schramm, R., et al. (2007) Transplantation 84, 555-558), to our knowledge, this study is the first to show utility of all three markers as cross-organ AR protein biomarkers in human serum or plasma.

TABLE 5
Patient demographics of cardiac transplant in ELISA study.

| | | | |
|---|---|---|---|
| Clinical Characteristics | AR (n = 32) | STA (n = 31) | P value |

Recipients

| | | | |
|---|---|---|---|
| Gender (% females) | 28% | 19% | 0.41 |
| Mean age (year) | 40.6 ± 16.3 | 46.1 ± 19.3 | 0.22 |
| Age range (year) | 11-66 | 10-69 | |
| Diastolic blood pressure | 78.3 ± 9.1 | 77.7 ± 17.8 | 0.87 |
| Systolic blood Pressure | 124.8 ± 14.0 | 125.0 ± 26.7 | 0.98 |
| Weight (kg) | 92.8 ± 21.6 | 83.1 ± 24.6 | 0.1 |
| Sample collection time | 10.0 ± 9.8 | 9.2 ± 5.4 | 0.71 |
| (month, post-transplant) | | | |
| Sample collection time range | 0.4-41 | 0.8-18 | |
| (month) | | | |
| Race (1, 2, 3, 4, 5)* | 69%, 9%, 6%, | 74%, 13%, | 0.82 |
| | 16%, 0% | 3%, 10%, 0% | |
| ESRD (1, 2, 3, 4, 5, 6)** | 59%, 6%, | 71%, 3%, | 0.5 |
| | 22%, 13% | 23%, 3% | |

Donors

| | | | |
|---|---|---|---|
| Gender (% females) | 22% | 19% | 0.8 |
| Age (year) | 31.0 ± 16.7 | 29.1 ± 18.8 | 0.56 |
| Age range (year) | 14-55 | 9-60 | |

Values are mean ± SD (Standard Deviation).
AR: Acute Rejection;
STA: stable;
ESRD: End stage renal disease.
Race (*) is defined as: 1 = Caucasian; 2 = Hispanic; 3 = Asian; 4 = African American; 5 = Other.
ESRD (**) categories are: 1 = Glomerulonephritis; 2 = Polycystic Kidney Disease; 3 = Dysplasia; 4 = Reflux Nephropathy; 5 = Obstructive Uropathy; 6 = Other.

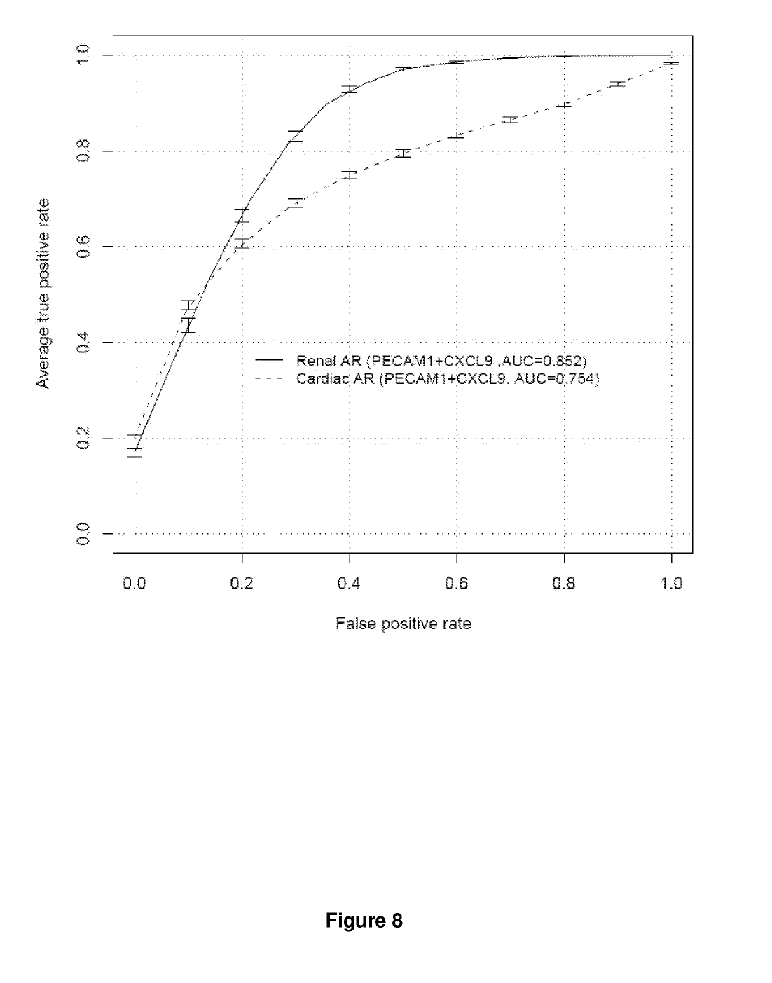

We evaluated the performance of a combined panel of PECAM1 and CXCL9 using a three-fold cross-validation. We randomly selected two third of samples, trained a multinomial logistic regression model, and calculated the predictive performance on the remaining one third of samples. After repeating the process 1000 times, the average ROC curves showed a slight improvement on cardiac AR and no improvements on renal AR (FIG. 8), suggesting a large clinical trial combing PECAM1 and CXCL9 with other previously found protein biomarkers would be needed for the accurate diagnosis of AR.

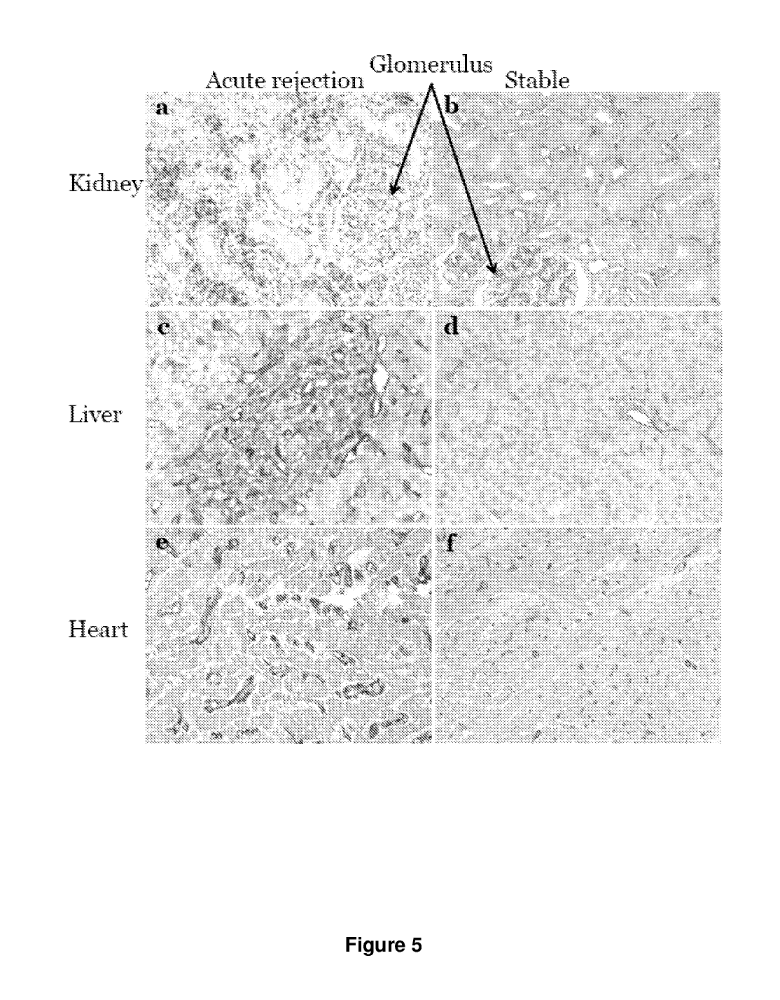

We performed an immunohistochemistry study on our best performing marker, PECAM1, to compare its protein expression between AR and STA in renal, hepatic and cardiac allograft biopsies (FIG. 5). In STA kidney tissue, PECAM1 staining was mainly observed in the endothelial cells of glomeruli, peritubular capillaries and large blood vessels. AR biopsies in addition revealed dense infiltrates of PECAM1 positive lymphocytes and mononuclear cells in the interstitium. Similarly, dense endothelial PECAM1 staining was observed in the hepatic and cardiac transplant AR tissues along with staining in infiltrating mononuclear cells, while only minor endothelial staining was found in hepatic and cardiac STA tissues. These immunohistochemistry results showed significantly increased PECAM1 protein expression in the AR tissues compared to STA tissues across transplanted organs.

Furthermore, our studies showed that PECAM1 protein was also statistically significantly upregulated in the serum samples from AR patients compared with samples from patients with BK virus infection and chronic rejection after renal transplants. Analysis across hundreds of diseases using our GeneChaser tool (Chen, R., et al. (2008) BMC Bioinformatics 9, 548) indicates that the mRNA expression of PECAM1 was significantly upregulated in various cancers and Parkinson's disease but not in any potential confounding conditions, such as infection and hypertension. Indeed, PECAM1 was significantly downregulated in hypertension (pfp=0.041, fold=0.845, Rank Product). These results suggested that PECAM1 is a serum marker specific for allograft acute rejection irrespective of the transplanted organs.

Finally, among the 45 cross-organ AR genes, twenty-three of our 45 AR genes in our common AR signature were involved in a single pro-inflammatory pathway regulated by STAT-1 (see FIG. 7). Among the ten proteins we tested by ELISA, five were within this pathway and five were outside of it. All five proteins outside the pathway failed validation, while three out of five proteins inside the pathway were validated as AR markers. The 60% success rate from this single pathway suggests that it is likely to be a common functional pathway in AR across organs being transplanted. More novel AR protein markers are likely to be found from the remaining 18 commonly upregulated proteins inside this pathway not yet tested by ELISA (see FIG. 7), include CD2, Cathepsin S (CTSS), and SH2D2A.

### III. Discussion

We developed a novel method to search for diagnostic protein biomarkers through integrating gene expression data in the disease tissues with biofluid proteome databases, and demonstrated the enrichment of clinically and pre-clinically validated protein biomarkers in 22 diseases. We applied our method to new and publicly-available measurements on solid-organ transplantation, and identified and validated three cross-organ serum protein biomarkers for transplant rejection. Our results demonstrated that integration of gene expression microarray measurements from disease samples, and especially publicly-available data sets, can be a powerful, fast, and cost-effective strategy for the discovery of new diagnostic serum biomarkers.

We found that the likelihood of finding protein biomarkers indicative for a disease increases with more gene expression data sets. Many meta-analysis methods have previously been shown to improve the identification of differentially expressed genes (Hong, F. & Breitling, R. A (2008) Bioinformatics 24, 374-382). The identification of protein biomarkers could be improved through sophisticated meta-analysis methods, such as Rank Product (Breitling, R., et al. (2004) FEBS Lett 573, 83-920), measurements of concordance among data sets (Lai, Y., et al. (2007) Bioinformatics 23, 1243-1250), and the identification of common features for diagnostic protein biomarkers.

It is evident that the subject invention provides a convenient and effective way of monitoring an allograft recipient for a rejection response without invasive means. As such, the subject invention provides a number of distinct benefits, including the ability to easily identify subjects undergoing allograft rejection, so that these individuals can be treated accordingly. As such, the subject invention represents a significant contribution to the art.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method for monitoring an allograft recipient for a rejection response, said method comprising:
evaluating the level of at least one protein in a sample from said allograft recipient to obtain a protein level determination; and
employing said protein level determination to monitor said allograft recipient for a rejection response.

2. The method of claim 1, wherein said sample is selected from serum, urine, blood, CSF, tears, and saliva.

3. The method of claim 2, wherein said protein is selected from the group consisting of: PECAM1 and CD44.

4. The method of claim 1, wherein said sample is serum.

5. The method of claim 4, wherein said protein is selected from the group consisting of: CXCL9, PECAM1 and CD44.

6. The method of claim 1, wherein said rejection response is an acute rejection.

7. The method according to claim 1, wherein said employing step comprises comparing said protein level determination to a reference profile.

8. The method according to claim 7, wherein said reference profile is selected from: a protein level determination from an individual that has not received an allograft; a protein level determination from a stable allograft recipient; and a protein level determination from an allograft recipient undergoing a rejection response.

9. A system for monitoring an allograft recipient for a rejection response, said system comprising:
(a) a protein level determination element for measuring the level of at least one protein in a sample from said allograft recipient to obtain a protein level determination; and
(b) a phenotype determination element for employing said protein level determination to monitor said allograft recipient for a rejection response.

10. The system according to claim 9, wherein said protein level determination element comprises at least one reagent for assaying a sample for said at least one protein.

11. The system according to claim 9, wherein said sample is selected from serum, urine, and blood.

12. The system according to claim 9, wherein said at least one protein is selected from CXCL9, PECAM1 and CD44.

13. The system according to claim 9, wherein said rejection response is an acute rejection.

14. A kit for monitoring an allograft recipient for a rejection response, said kit comprising:
(a) a protein level determination element for measuring the level of at least one protein in a sample from said allograft recipient to obtain a protein level determination; and
(b) a phenotype determination element for employing said protein level determination to monitor said allograft recipient for a rejection response.

15. The kit according to claim 12, wherein said protein level determination element comprises at least one reagent for evaluating a sample for said at least one protein.

16. The kit according to claim 12, wherein said sample is selected from serum, urine, and blood.

17. The kit according to claim 12, wherein said at least one protein is selected from CXCL9, PECAM1 and CD44.

18. The kit according to claim 16, wherein said rejection response is an acute rejection.

19. A method of identifying a protein biomarker for allograft rejection, said method comprising:
obtaining gene expression profiles from a plurality of allograft recipients undergoing a rejection response, wherein said plurality of allograft recipients comprises recipients for at least two different tissue types and said gene expression profiles comprise gene expression profiles derived public databases;
identifying genes that are upregulated in all of said plurality of allograft recipients;
evaluating whether protein levels for said identified genes are elevated in a sample from allograft recipients undergoing a rejection response.


SEQUENCE LISTING (14 sequences)

SEQ ID NO: 1 - DNA, 3754 bp, homo sapiens
ccaggccccattgttcccggtttccagccatggctgccattacctgaccagcgccacagccggtctctctgcaggcgccgggagaagtgaccagagcaatttctgcttttcacagggcgggtttctcaacggtgacttgtgggcagtgccttctgctgagcgagtcatggcccgaaggcagaactaactgtgcctgcagtcttcactctcaggatgcagccgaggtgggcccaaggggccacgatgtggcttggagtcctgctgacccttctgctctgttcaagccttgagggtcaagaaaactctttcacaatcaacagtgttgacatgaagagcctgccggactggacggtgcaaaatgggaagaacctgaccctgcagtgcttcgcggatgtcagcaccacctctcacgtcaagcctcagcaccagatgctgttctataaggatgacgtgctgttttacaacatctcctccatgaagagcacagagagttattttattcctgaagtccggatctatgactcagggacatataaatgtactgtgattgtgaacaacaaagagaaaaccactgcagagtaccagctgttggtggaaggagtgcccagtcccagggtgacactggacaagaaagaggccatccaaggtgggatcgtgagggtcaactgttctgtcccagaggaaaaggccccaatacacttcacaattgaaaaacttgaactaaatgaaaaaatggtcaagctgaaaagagagaagaattctcgagaccagaattttgtgatactggaattccccgttgaggaacaggaccgcgttttatccttccgatgtcaagctaggatcatttctgggatccatatgcagacctcagaatctaccaagagtgaactggtcaccgtgacggaatccttctctacacccaagttccacatcagccccaccggaatgatcatggaaggagctcagctccacattaagtgcaccattcaagtgactcacctggcccaggagtttccagaaatcataattcagaaggacaaggcgattgtggcccacaacagacatggcaacaaggctgtgtactcagtcatggccatggtggagcacagtggcaactacacgtgcaaagtggagtccagccgcatatccaaggtcagcagcatcgtggtcaacataacagaactattttccaagcccgaactggaatcttccttcacacatctggaccaaggtgaaagactgaacctgtcctgctccatcccaggagcacctccagccaacttcaccatccagaaggaagatacgattgtgtcacagactcaagatttcaccaagatagcctcaaagtcggacagtgggacgtatatctgcactgcaggtattgacaaagtggtcaagaaaagcaacacagtccagatagtcgtatgtgaaatgctctcccagcccaggatttcttatgatgcccagtttgaggtcataaaaggacagaccatcgaagtccgttgcgaatcgatcagtggaactttgcctatttcttaccaacttttaaaaacaagtaaagttttggagaatagtaccaagaactcaaatgatcctgcggtattcaaagacaaccccactgaagacgtcgaataccagtgtgttgcagataattgccattcccacgccaaaatgttaagtgaggttctgagggtgaaggtgatagccccggtggatgaggtccagatttctatcctgtcaagtaaggtggtggagtctggagaggacattgtgctgcaatgtgctgtgaatgaaggatctggtcccatcacctataagttttacagagaaaaagagggcaaacccttctatcaaatgacctcaaatgccacccaggcattttggaccaagcagaaggctagcaaggaacaggagggagagtattactgcacagccttcaacagagccaaccacgcctccagtgtccccagaagcaaaatactgacagtcagagtcattcttgccccatggaagaaaggacttattgcagtggttatcatcggagtgatcattgctctcttgatcattgcggccaaatgttattttctgaggaaagccaaggccaagcagatgccagtggaaatgtccaggccagcagtaccacttctgaactccaacaacgagaaaatgtcagatcccaatatggaagctaacagtcattacggtcacaatgacgatgtcagaaaccatgcaatgaaaccaataaatgataataaagagcctctgaactcagacgtgcagtacacggaagttcaagtgtcctcagctgagtctcacaaagatctaggaaagaaggacacagagacagtgtacagtgaagtccggaaagctgtccctgatgccgtggaaagcagatactctagaacggaaggctcccttgatggaacttagacagcaaggccagatgcacatccctggaaggacatccatgttccgagaagaacagataatccctgtatttcaagacctctgtgcacttatttatgaacctgccctgctcccacagaacacagcaattcctcaggctaagctgccggttcttaaatccatcctgctaagttaatgttgggtagaaagagatacagaggggctgttgaatttcccacataccctccttccaccaagttggaacatccttggaaattggaagagcacaagaggagatccagggcaaggccattgggatattctgaaacttgaatattttgttttgtgcagagataaagaccttttccatgcaccctcatacacagaaaccaattttcttttttatactcaatcatttctagcgcatggcctggttagaggctggttttttctcttttcctttggtccttcaaaggcttgtagttttggctagtccttgttctttggaaatacacagtgctgaccagacagcctccccctgtcccctctatgacctcgccctccacaaatgggaaaaccagactacttgggagcaccgcctgtgaaataccaacctgaagacaccgttcattcaggcaacgcacaaaacagaaaatgaaggtggaacaagcacagatgttcttcaactgtttttgtctacactctttctcttttcctctaccatgctgaaggctgaaagacaggaagatggtgccatcagcaaatattattcttaattgaaaacttgaaatgtgtatgtttcttactaatttttaaaaatgtattccttgccagggcaggcaaggtggctcacgcctgtaatcccagcacttcaggaggctgaggtgggcggatcacctgaggtcaggagtttgagaccagcctgatgaaaccctgtctctactaaaaatacaagaattagccgggcgtggtggcgcatgcctgtagtatcagctactcaagaggctgaggtgagattatcgcttgaacccaggaaacggaggttgtagtgagcggagatcgcgccactgcactccagcctgagtgacagagtgagaatccatctcaaaaaaaacaaaaaacaaaattgcttgctaaagaagtggtctcctgaggtcttaagacattcctgacagtgtcttgagtgggtgggagagaggctgctgtcattgcgctgtggaatttcacagatgagaaccacgcctagccaaaatcacttttcctgtttgcctcagtgacacagctgcagggaccctcgtggatgttgtattaaataaatttgacctttgctctttgcaaaaaaaaaaaa

SEQ ID NO: 2 - protein, 738 aa, homo sapiens
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQLLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 3 - DNA, 2545 bp, homo sapiens
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 4 - protein, 125 aa, homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 5 - DNA, 5748 bp, homo sapiens
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 6 - protein, 742 aa, homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 7 - DNA, 5619 bp, homo sapiens
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 8 - protein, 699 aa, homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATSTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 9 - DNA, 5001 bp, homo sapiens
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccaatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 10 - protein, 493 aa, homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 11 - DNA, 4605 bp, homo sapiens
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 12 - protein, 361 aa, homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 13 - DNA, 3985 bp, homo sapiens
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 14 - protein, 139 aa, homo sapiens
MDKFWWHAAWGLCLVPLSLAQIDLNITCRFAGVFHVEKNGRYSISRTEAADLCKAFNSTLPTMAQMEKALSIGFETCSLHCSQQSKKVWAEEKASDQQWQWSCGGQKAKWTQRRGQQVSGNGAFGEQGVVRNSRPVYDS